(19) United States
(12) Patent Application Publication
(10) Pub. No.: US 2010/0285463 A1
(43) Pub. Date: Nov. 11, 2010

(54) SELECTING NUCLEIC ACID SAMPLES SUITABLE FOR GENOTYPING

(75) Inventors: Florian Kronenberg, Innsbruck (AT); Veit Schoenborn, Kempten (DE); Iris Heid, Regensburg (DE); Anita Brandstätter, Innsbruck (AT)

Correspondence Address: FULBRIGHT & JAWORSKI L.L.P., 600 CONGRESS AVE., SUITE 2400, AUSTIN, TX, 78701, US

(73) Assignee: Medizinische Univversitat Innsbruck, Innsbruck (AT)

(21) Appl. No.: 12/667,135

(22) PCT Filed: Jun. 27, 2008
(86) PCT No.: PCT/EP08/05280

§ 371 Date: Jun. 23, 2010

(30) Foreign Application Priority Data
Jun. 29, 2007 (EP) 07012858.2

(51) Int. Cl. C12Q 1/68

(52) U.S. Cl. 435/6

(57) ABSTRACT

The present invention relates to a method for selecting a DNA sample comprising genomic DNA suitable for genotyping, comprising the steps of: i) pre-genotyping said genomic DNA using a set of polymorphic markers; ii) determining out of said set of polymorphic markers the percentage of polymorphic markers for which said genomic DNA is homozygous; and iii) selecting said DNA sample when said genomic DNA is homozygous for less than 70% of said set of polymorphic markers. Furthermore, a method of genotyping comprising a step of using a DNA sample selected by the method in accordance with the present invention and/or a step of applying the method provided herein is disclosed. The present invention also relates to a method for identifying a gene or a locus on a genome, said method comprising a step of using a DNA sample selected by the method provided herein and a step of applying the method described herein. Further, the present invention relates to a kit for carrying out the method in accordance with the present invention comprising primers for the amplification of the set of polymorphic markers employed herein.

The present invention relates to a method for selecting a nucleic acid sample, in particular a DNA sample comprising genomic DNA suitable for genotyping, comprising the steps of: (i) pre-genotyping said genomic DNA using a set of polymorphic markers; (ii) determining out of said set of polymorphic markers the percentage of polymorphic markers for which said genomic DNA is homozygous; and (iii) selecting said DNA sample when said genomic DNA is homozygous for less than 70% of said set of polymorphic markers. Furthermore, a method of genotyping comprising a step of using a DNA sample selected by the method in accordance with the present invention and/or a step of applying the method provided herein is disclosed. The present invention also relates to a method for identifying a gene or a locus on a genome, said method comprising a step of using a DNA sample selected by the method provided herein and/or a step of applying the method described herein. Further, the present invention relates to a kit for carrying out the method in accordance with the present invention comprising primers for the amplification of the set of polymorphic markers to be employed herein.

For classical long-standing cohort epidemiological studies, numerous biological samples and data on incident events had been collected over decades with more than 10000 individuals in some studies minimizing major biases such as survival bias (see Dawber (1966), Circulation 34, 553-555; Belanger (1978), Am J Nurs 78, 1039-1040). A major disadvantage is typically the availability of only plasma or serum samples only for analysis of biomarkers. The absence of whole blood or buffy coat makes standard genotype analysis impossible. In other studies, whole blood DNA is only available for survivors, which is only of limited help for investigating genetic factors for survival. Therefore, important information is not accessible to scientific analysis.

Plasma or serum samples usually have small amounts of DNA since most of the cells have been removed. Further, the quality of DNA derived from plasma or serum samples can be low since DNA may be freely floating in the sample and may therefore be prone to degradation. Long-term storage of samples may also reduce the concentrations of high molecular weight DNA in the sample and decrease the quality of the DNA.

Whole genome amplification (WGA) can be a powerful tool to recover genomic DNA, which is available in minute amounts in plasma and serum samples, see Blomeke (1997), Carcinogenesis 18, 1271-1275; Stemmer (2003), Clin Chem 49, 1953-1955. During the last 15 years, several PCR-based methods have been developed for WGA, see Telenius (1992), Genomics 13, 718-725; Cheung (1996), Proc Natl Acad Sci USA 93, 14676-14679; Grant (2002), Nuc Acids Res 30, e125; Zhang (1992), Proc Natl Acad Sci USA 89, 5847-5851; Dietmaier (1999), Am J Pathol 154, 83-95. However, WGA is known to produce nonspecific amplification artefacts, see Cheung (1996, loc. cit.), to give incomplete coverage of loci, see Dean (2002), Proc Natl Acad Sci USA 99, 5261-5266; Paunio (1996), Clin Chem 42, 1382-1390, and to generate short DNA fragments (<3 kb), see Telenius (1992; loc. cit.). This may lead to large genotype error and limits their use.

Recently, a new WGA method, multiple displacement amplification (MDA), was developed to amplify DNA in a hyperbranching, isothermal reaction. It uses the highly processive φ29 DNA polymerase and random exonuclease-resistant primers to amplify the entire genome with a 10000-fold amplification and achieves DNA fragments with >10 kb in length, see Dean (2002; loc. cit.). This enzyme combines a high proofreading activity resulting in low error rates with an unbiased amplification of the original genome, see Hosono (2003), Genome Res 13, 954-964. MDA performance was demonstrated for a variety of applications including SNP and STR analysis, see Hosono (2003; loc. cit.); Barker (2004), Genome Res 14, 901-907; Paez (2004), Nucleic Acids Res 32, e71; Faruqi (2001), BMC Genomics 2:4; Dickson (2005), Nucleic Acids Res 33, e119; Matsuzaki (2004), Genome Res 14, 414-425; Lovmar (2003), Nucleic Acids Res 31, e129; Holbrook (2005), J Biomol Tech 16, 125-133, restriction fragment length polymorphism analysis (RFLP), and comparative genomic hybridization (CGH), see Dean (2002; loc. cit.); Lage (2003), Genome Res 13, 294-307. In the meantime it enjoys a widespread routine application especially for the reproduction of available but limited DNA samples. However, recent data pointed towards the possibility of allelic dropouts in different samples due to insufficient DNA quality. For example, allelic drop-outs can occur after WGA due to the very low amount of DNA in plasma or serum samples, see Bergen (2005), BMC Biotechnol 5:24. Increasing the amount of DNA to improve genotyping results may not be an option if only limited amounts of DNA are available.

Accordingly, there is a need for reliable DNA genotyping, in particular when DNA samples are used for genotyping which are derived from biological samples characterised by small amounts and/or low quality of genomic DNA, such as plasma or serum samples. Further, it is well known from statistical methodology that genotyping error induces biased association estimates mostly yielding an underestimation of effects, see Carroll (1995), Chapman & Hall/CRC. Considering the enormous value of the longitudinal long-standing cohort studies without banked. DNA of part or all of the participants makes a restriction to samples with reliable WGA plasma-DNA mandatory.

Sjöholm (2005) shows that whole genomic DNA from plasma or serum samples may be amplified by MDA and used for subsequent genotyping such as TaqMan Assays, see Sjöholm (2005), Cancer Epidemiol Biomarkers Prey 14, 251-255. Genotyping results were considered successful either if identical results were obtained twice or if identical results were obtained compared to genomic DNA extracted from tissue of the same subject. The tissue samples used as standard had been fixed in formalin and stored for several years, similar to the plasma and serum samples which had been collected since 1978. Therefore, the comparison of genotyping results using genomic DNA before whole genome amplification (WGA) and after WGA rather reflects the reliability of amplification of whole genomic DNA as such. However, the results obtained do not reflect the reliability of the DNA samples itself. Thus, Sjöholm does not teach that the DNA used for genotyping before or after WGA of the DNA has to be of sufficient quality to give reliable genotyping results. Rather, Sjöholm suggests the use of more than 0.2 ng of genomic DNA as template for WGA and does not mention that also the DNA quality of a sample essentially influences the outcome of a genotyping assay.

Lu (2005) describes a method of SNP genotyping using genomic DNA extracted from plasma samples, see Lu (2005), Biotechniques 39, 511-515. Similar to Sjöholm (2005, loc. cit.) Lu compares the results of SNP genotyping using DNA samples from the same subject before and after WGA. As outlined above, the results obtained may reflect the reliability of WGA but not the quality of the DNA sample as such. Lu proposes that DNA samples the whole genomic DNA of which is not amplified are not suitable for genotyping. Further, Lu attributes the failure rate of genotyping using DNA samples before WGA to the low yield of genomic DNA but not to low quality of said genomic DNA.

Lu (2005; loc. cit.) and Sjöholm (2005, loc. cit.) observed discordances to a relatively low extent. This is probably caused by the relatively lower number of SNPs they investigated or due to the possibility that the failure is already present in the starting material, namely the plasma DNA. Such an experimental design therefore might only detect the effect of WGA, but not the problems of the starting material.

Bashiardes (2006) also investigates the reliability of whole genome amplification, in particular MDA, by quantification analysis of selected loci of genomic DNA extracted from 1 respectively 10 lymphocytes compared to unamplified genomic DNA, see Bashiardes (2006), Clin Chem Lab Med 44, 1158-1160. He speculates that the amount of starting template for amplification of whole genomic DNA may be a decisive factor for the quality of the amplified product. He further hypothesizes that the lack of efficient cell lysis may prevent genomic DNA from being released for access to the polymerase used in the amplification process. According to Bashiardes, efficient cell lysis may be of particular relevance when genomic DNA of single cells is amplified.

To summarize, neither Sjöholm (2005, loc. cit.), Lu (2005; loc. cit.) nor Bashiardes (2006; loc. cit.) describe or suggest a method for identifying DNA samples with sufficient quality for genotyping. Instead, the reliability of WGA as such is evaluated using genomic DNA from plasma samples and amplified whole genomic DNA of the same sample respectively genomic DNA from lymphocytes and amplified whole genomic DNA from single lymphocytes. One may deduce from the teaching provided in the literature cited above that increasing the amount of input DNA may lead to improved results obtained in the amplification process.

Dickson (2005), Bergen (2005) and Lovmar (2003) have compared genotyping results of amplified whole genomic DNA with genotyping results of unamplified genomic DNA used as standard, see Dickson (2005; loc. cit.); Bergen (2005; loc. cit.); Lovmar (2003: loc. cit.). Bergen speculates that amplified whole genomic DNA may not be suitable for STR genotyping at all. In particular, STR genotyping of WGA-DNA derived from 1 ng genomic input DNA showed a discordance rate as low as 80%. Increasing the amount of genomic input DNA up to 200 ng was followed by an increase of concordance rate to about 99%. However, in case the available amount of genomic input DNA is limited, one may not be able to provide a sufficient amount of genomic DNA in order to obtain such a concordance rate. Further, Bergen shows that at least 10 ng genomic input DNA into the WGA reaction has to be used in order to obtain performance rates in SNP genotyping similar to unamplified genomic DNA. Thus, Bergen clearly teaches that an increase in genomic input DNA into the WGA reaction improves the genotyping performance.

Lovmar (2003; loc. cit.) proposes to use at least 0.3 ng of genomic input DNA into the WGA reaction in order to obtain reliable SNP genotyping results, while 3 ng of genomic input DNA should give the most reproducible results. Lovmar states that the variation in the amplification of SNP alleles depends on the amount of genomic input DNA.

Dickson (2005; loc. cit.) investigated the applicability of MDA in STR genotyping. He proposes to pool WGA-DNA obtained from replicate amplification reactions using the same genomic input DNA in order to improve the concordance rate. Further, Dickson aims at optimizing STR sets used for genotyping of amplified whole genomic DNA.

In the prior art, the need for improving the quality of genotyping is recognized, in particular in case whole genomic DNA is amplified prior to genotyping. Yet, the above mentioned publications propose an increase of genomic input DNA into the WGA reaction or the use of an optimized marker set in order to improve the reliability of genotyping.

Thus, the technical problem underlying the present invention is the provision of means and methods for the identification of DNA samples with sufficient quality for increased the reliability in genotyping and other molecular assessments

The technical problem is solved by provision of the embodiments characterized in the claims.

Accordingly, the present invention relates to a method for selecting a DNA sample comprising genomic DNA suitable for genotyping, comprising the steps of:
(i) pre-genotyping said genomic DNA using a set of polymorphic markers;
(ii) determining out of said set of polymorphic markers the percentage of polymorphic markers for which said genomic DNA is homozygous; and
(iii) selecting said DNA sample when said genomic DNA is homozygous for less than 70% of said set of polymorphic markers.

In accordance with the present invention, the term “genomic DNA” in particular relates to DNA that is derived from a genome. The term, however, also encompasses RNA molecules (like genomic (g) RNA or nucleolar(n) RNA as well as non-spliced or partially spliced RNA) that may be reverse transcribed into DNA in accordance to standard methods

In an alternative embodiment, the present invention relates to said method for selecting a DNA sample comprising genomic DNA suitable for genotyping, comprising the steps of: (i) pre-genotyping said genomic DNA using a set of polymorphic markers, (ii) determining out of said set of polymorphic markers the percentage of polymorphic markers for which said genomic DNA is homozygous, and (iii) selecting said DNA sample when said genomic DNA is homozygous for less than 60% of said set of polymorphic markers.

The present invention solves the above identified technical problem since, as documented herein below and in the appended example, it was surprisingly found that the method provided herein of selecting DNA samples is much more effective in increasing the reliability of genotyping as compared to known selection methods while, at the same time, less samples are excluded. In contrast to the prior art, the method of the present invention relates to the selection of DNA samples prior to genotyping without the need of increasing the amount of input DNA or adapting the markers used for genotyping. The present invention allows a much more reliable assessment of biological samples a well as forensic samples.

Another surprising finding was that the reliability of genotyping can be strongly increased by identifying and selecting DNA samples comprising genomic DNA with sufficient quality following a step of pre-genotyping these DNA samples. DNA with sufficient quality employed herein can be described as DNA which represents the original state of the DNA. The original state of the DNA may be, for example, non-degraded DNA comprised in biological material/organism described herein. Accordingly, low quality DNA employed herein can best be described as DNA which does not represent the original state of the DNA. This may be caused, for example, by degradation of the DNA or by unbalanced amplification of DNA. A person skilled in the art knows that, for example, blood plasma usually comprises low quality DNA while whole blood usually comprises DNA of high quality. Thus, DNA derived from whole blood may, for example, represent the original state of the DNA and serve as standard as shown in the appended example.

It is one main advantage of the selection method of the present invention that the selected DNA samples can be used in various and that these genotyping assays can be dissimilar to the assay employed in the step of pre-genotyping.

Furthermore, the method of the present invention allows for the selection of genomic DNA (gDNA) samples, like DNA samples amplified by whole genome amplification (WGA), which leads to an enormous increase in the reliability of genotyping, i.e. a decrease in the discordance rate at least by the factor 4. As shown in the appended example, the exclusion of about one fourth of the samples according to the method disclosed herein resulted in an pronounced decrease in the discordance rate by the factor 4 for STR (short tandem repeat) genotypes and by the factor 6,5 for SNP (single nucleotide polymorphism) genotypes. Thereby, the observed discordance rate for SNPs is very close to the error rate seen for genomic whole blood DNA in many laboratories, see Pompanon (2005), Nat Rev Genet. 6, 847-859. The surprisingly high usefulness of the selection method of the present invention is further supported by the observation that most of the STR and SNP markers which violated Hardy Weinberg equilibrium (HWE) before the selection process improved their HWE after exclusion of the non-reliable samples. Thereby, the rate of STR heterozygosity increased to a frequency similar to that in unamplified genomic DNA samples derived from e.g. whole blood or other samples that are to be genotyped, like forensic samples as well as clinical samples. The selection method provided herein provides for nucleic acid samples, i.e. samples that are of higher quality for molecular assessments (clinical as well as forensic samples) and genotyping than randomly selected samples,

Previous studies described a coherence between the amount of input DNA and genotyping performance, see Bergen (2005; loc. cit.); Lovmar (2003; loc. cit.). As shown herein and in the appended example, a method of selecting DNA samples with respect to the amount of input DNA which in the end depends on the DNA concentration of the plasma sample is disadvantageous. Such a selection method results in a higher sample rejection rate and, at the same time, in discordance rates about twice as high compared to the discordance rates obtained by applying the method of the present invention.

Pompanon discussed that low amounts of input DNA might induce allele amplification bias, see Pompanon (2005; loc. cit.). However it is not only the small amount of input DNA but also a low quality of the input DNA which may lead to relatively worse genotyping results, such as allele amplification bias. A potential degradation present in the plasma DNA before WGA can cause allelic drop-outs as shown recently, see Schneider (2004), Forensic Sci Int 139:123-134. An increase of allelic drop-outs is observed when degraded DNA is used as starting material for WGA, see Lasken (2003), Trends Biotechnol 21, 531-535; Ballantyne (2007), Forensic Sci Int. 166:35-41. WGA tends to amplify larger DNA fragments. Accordingly, in case the DNA collected from plasma contains substantial amounts of smaller DNA fragments, only a fraction is amplified by WGA which might increase the probability of allelic drop-outs. Whether the recently described blunt-end ligation-mediated method, see Li (2006), J Mol Diagn 8, 22-30, is more tolerant to sample degradation has to be determined.

Thus, the present invention provides for a method of selecting DNA with sufficient quality which is superior to a method based on the selection of DNA samples with respect to the DNA concentration/amount. One advantage of the method of the invention over said selection method is the fact that samples can be selected for reliable genotyping with a very low input of genomic DNA into the WGA reaction, for example an input of less than 0.1 ng; as shown in the appended example. On the one hand, an exclusion limit like that proposed in the prior art (0.2 ng genomic input DNA), see Sjöholm (2005; loc. cit.), would result in the loss of samples which are indeed suitable for genotyping. On the other hand, samples with an input DNA of more than 0.2 ng genomic DNA may not necessarily be suited for genotyping thus decreasing the overall genotyping performance.

As a further advantageous property, the method of the invention allows for the assessment of the quality of the starting material while methods known in the art only allow for an assessment of the reliability of the amplification of whole genomic DNA. Low quality DNA employed herein can best be described as DNA which does not represent the original state of the DNA. This may be caused, for example, by degradation of the DNA or by unbalanced amplification of DNA. Accordingly, the original state of the DNA may be, for example, non-degraded DNA comprised in biological material/organism described herein. A person skilled in the art knows that, for example, blood plasma usually comprises low quality DNA while whole blood usually comprises DNA of high quality. Thus, DNA derived from whole blood may, for example, represent the original state of the DNA and serve as standard as shown in the appended example.

Since the effect of both DNA amplification (like WGA) and starting material is included in the assessment of the outcome of genotyping assays, the method of the present invention may also be applicable for testing the reliability of a wide range of genomic DNA samples. Said genomic DNA samples may be samples containing DNA that is eventually degraded due to, inter alia, repeated thawing and freezing cycles, such containing DNA that has been stored for a long period of time or such having a low genomic DNA concentration. Also other nucleic acid samples, like forensic samples or old samples may be used as a source of DNA to be selected in accordance with the invention.

A further advantage of the method provided herein is the reduction in genotyping costs (in the long run), since the non-reliable DNA samples (about a fourth of the total samples) can be excluded from further genotyping, as shown in the appended example. Yet, and preferably, pre-genotyping DNA samples in order to find reliable samples is only performed once. The selected DNA samples may then be used for several subsequent genotyping experiments in that samples.

One major benefit from applying the method of the present invention is the reduction of genotyping errors. The statistical theory predicts that a genotyping error, which is not different between healthy subjects (“controls”) and subjects with disease (“cases”), i.e. non-differential error, induces a bias towards the null. In other words, a truly existing disease association with a given phenotype would be underestimated. In contrast, differential genotyping error, that is genotyping error different between cases and controls, should be avoided by all means, because the direction of bias is not predictable and either spurious associations or a bias towards the null can be induced. For example, DNA from whole blood may be available for all subjects in a follow-up (“controls” in survival analysis) cohort study, but DNA from the non-survivors (“events”) may have to be derived from baseline plasma using WGA. The lower quality DNA derived from WGA of some samples may induce larger genotyping error for the events as compared to the controls. Thus, the method disclosed herein may greatly alleviate this differential genotyping error by selecting only DNA samples with sufficient DNA quality.

Further, the method of the present invention does not only work as stand-alone, when no whole blood DNA is available, but it is, for example, also a valuable tool when assuring the comparability of WGA-derived DNA genotypes of a subsample with the whole blood DNA genotypes of the rest.

In general, the “DNA sample” to be selected in accordance with the method of the present invention may be derived from any biological source/organism, particularly any biological source/organism, the genome of which is intended to be genotyped. The DNA sample may be derived from a virus or a single- or multicellular organism.

In the context of the present invention, the term “virus” means a biological infectious particle which can only replicate itself by infecting a host cell. The infected host cell can be an animal cell as well as a plant cell. Such a virus may be, for example, herpes simplex virus, papillomavirus, borna virus, tobacco mosaic virus and T4 phage. Other clinically relevant viruses comprise hepatitis virus (HCV, HBV, HAV, HEV or other non-A-nonB-hepatitis virus), HIV and SIV, and the like. “Homo-” and “Heterozygosity” in context of this embodiment of the invention may also be seen in heterologous samples or heterologous cultures.

Generally, it is well known in the art that various organisms, for example those described herein and in particular viruses can be genotyped. Such organisms may be also be pre-genotyped and the corresponding nucleic acid samples, in particular DNA samples, selected in accordance with the present invention when heterogenous populations of these organisms exist (i.e. the population is heterozygous for a set of markers (or subgroup thereof) as defined herein). A person skilled in the art will be aware of corresponding means and methods for genotyping/pre-genotyping such samples and will also know corresponding polymorphic markers to be used in said genotyping/pre-genotyping.

Said single- or multicellular organism may be selected from the group consisting of bacteria, protists, fungi, plants and animals. The meaning of these terms is well known in the art. Again, also pathogenic organisms are comprised and their nucleic acid samples may be selected according to the present method.

In the context of the present invention, the term “bacteria” particularly means prokaryotes comprising the evolutionary domains Bacteria and Archaea. Examples for such bacteria are Neisseria sp., Streptococcus sp., Staphylococcus sp., Actinobacteria, and Escherichia coli. Other pathogenic bacteria may, for example, comprise Listeria species.

In the context of the present invention the term “protist” particularly means single- to few-cellular eukaryotes. Particular “protists” are, for example, Euglena sp., Amoeba sp., Paramecium sp., Toxoplasma sp., Ulva sp., Porphyra sp., and Macrocystis sp. The term, therefore, also comprises pathogenic protists.

The DNA sample to be selected may also be derived from fungi. The meaning of the term “fungi” is known by the skilled person and is used accordingly in the context of the present invention. In this context, the term “fungi” means, for example, heterotrophic eucaryotes which digest their food externally, which are not able to perform photosynthesis and which usually have cell walls. Examples for “fungi” are Penicillium sp., Agaricus sp., Phytophtora sp. and Amanita sp. Pathogenic fungi are comprised and their nucleic acid samples may be selected in accordance with the present selection method.

The DNA sample may also be derived from plants. In the context of the present invention the term “plant” particularly means phototrophic eucaryotes which comprise algae, bryophytes, ferns and higher plants such as gymnosperms and angiosperms. Plants to be used include but are not limited to maize, wheat, potato, tomato, tobacco and thale cress (Arabidopsis thaliana).

Yet, and preferably, the DNA sample to be selected is derived from an animal. More preferably, said DNA sample is derived from a mammal. The meaning of the terms “animal” or “mammal” is well known in the art and can, for example, be deduced from Wehner und Gehring (1995; Thieme Verlag). In the context of the present invention, the term “animal” particularly means a eucaryotic, heterotrophic organism which lacks cell walls and which usually digests food in an internal chamber. In the context of the present invention “mammal” particularly means a vertebrate, warm-blooded animal which is characterized by the production of milk in the female mammary glands. Non-limiting examples for mammals are even-toed ungulates such as sheep, cattle and pig, odd-toed angulates such as horses as well as carnivors such as cats and dogs. In the context of this invention, it is particularly envisaged that DNA samples are derived from organisms that are economically, agronomically or scientifically important or pose a possible threat to human health or the environment. Scientifically important organisms include, but are not limited to, mice, rats, rabbits, fruit flies like Drosophila melagonaster and nematodes like Caenorhabditis elegans.

The DNA sample may also be derived from primates which comprise lemurs, monkeys and apes. The meaning of the terms “primate”, “lemur”, “monkey” and “ape” is known and may, for example, be deduced by an artisan from Wehner und Gehring (1995, Thieme Verlag). In the context of the present invention the term “primate” means mammals that have five fingers, a generalized dental pattern, an unspecialized body plan, opposing thumbs and fingernails. “Primates” are, for example, Pongo sp., Gorilla sp. and Pan sp.

However, most preferably, the DNA sample is derived from a human being. The person skilled in the art is aware of the meaning of the term “human” and “human being”, and the like.

Generally spoken, the DNA sample to be selected in context of this invention may be derived from any kind of organic matter comprising genomic DNA. Said organic matter is preferably derived from living organisms, but it may, for example, also be derived from corpses, in particular human corpses.

Each specific, nucleic acid sample, in particular a DNA sample, which is to be selected in accordance with this invention, is derived from genomic DNA. The source of the genomic DNA to be tested can be any biological, medical/clinical or forensic sample. Examples of medical and forensic samples include blood, semen, vaginal swabs, tissue, hair, saliva, urine and mixtures of body fluids. These samples can be fresh, old, dried and/or partially-degraded. The samples can also be collected during sample taking by a medical personal or can be derived from evidence at a scene of a crime.

The term “forensic sample” as used herein means using the technology for legal problems including but not limited to criminal, paternity testing and mixed-up samples. The term “medical sample” as used herein means use of the technology for medical problems including but not limited to research, diagnosis, and tissue and organ transplants.

The means and methods provided herein and relating to the assessment of nucleic acid samples, in particular genomic DNA samples in genotyping/DNA typing can, inter alia, be employed in techniques for the determination of disease status or genetic constitution of a patient or in techniques for determining the relationship between given nucleic acid molecules, i.e. two or more genomic DNA samples. Applications of such a DNA typing may also comprise paternity testing and forensic science, and sample source determinations in transplantation, prenatal as well as post-natal diagnosis, or pedigree validation. Also the appended examples provide for working examples of genotyping events, like STR genotyping, MALDI-TOF SNP genotyping, TaqMan SNP genotyping and the like.

In a preferred embodiment of the present invention the DNA sample is derived from or is (a) cell(s), (a) tissue(s) or (a) body fluid(s). It is particularly envisaged that the cell, tissue or body fluid is derived from any one of the single- or multicellular organisms described herein. The DNA sample may be derived from a single cell, a plurality of cells and a tissue. The term “plurality of cells” means in the context of the present invention a group of cells comprising more than a single cell. Thereby, the cells out of said group of cells may have a similar function. Said cells may be connected cells and/or separate cells. The term “tissue” in the context of the present invention particularly means a group of cells that perform a similar function.

Examples for plant tissues are epidermis, vascular tissue and ground tissue. The term “plantal epidermis” in the context of the present invention means cells forming the outer surface of the leaves and of the young plant body. In the context of the present invention, the term “vascular tissue” means the primary components of vascular tissue, namely xylem and phloem. The term “ground tissue” means in the context of the present invention less differentiated tissue which performs photosynthesis and stores reserve nutrients.

Non-limiting examples for animal tissues are epithelium, connective tissue, muscle tissue and nervous tissue. The meaning of the terms “epithelium”, “muscle tissue”, “nervous tissue” and “connective tissue” are well known in the art. In the context of this invention “epithelium” particularly means tissues composed of layers of cells that cover organ surfaces such as surface of the skin and inner lining of digestive tract. The term “muscle tissue” particularly means in the context of the present invention muscle cells which contain contractile filamen. Muscle tissue can be part of a smooth muscle, which is found in the inner linings of organs; part of a skeletal muscle, which is found attached to bone; or part of a cardiac muscle found in the heart. In the context of the present invention, the term “nervous tissue” particularly means a tissue comprising cells which form parts of the brain, spinal cord and peripheral nervous system. The term “connective tissue” particularly means in the context of the present invention a tissue which is involved in structure and support. Examples for connective tissue are blood, cartilage and bone. The cells and tissues to be employed in accordance with the present invention may also be cultured cells or tissues.

In the context of the present invention the term “body fluid”, for example, means a fluid that is secreted or excreted from an animal or human body. However, a “body fluid”, for example, of human or animal origin, may also normally not be excreted or secreted. It is envisaged herein that the DNA sample can be derived from any body fluid or other parts of the body that has the chance of comprising at least one single cell, traces of cells, for example cell debris, or genomic DNA, even though the DNA may be degenerate or present in minute amounts. Non-limiting examples of body fluids are selected from the group consisting of amniotic fluid, aqueous humour, bile, cerumen, cowper's fluid, chyle, chyme, female ejaculate, interstitial fluid, lymph, menses, breast milk, mucus, pleural fluid, pus, saliva, sebum (skin oil), semen, sweat, tears, urine, vaginal lubrication, vomit, feces, cerebrospinal fluid, synovial fluid, intracellular fluid, and vitreous humour (fluid in the eyeball). Preferably, the body fluid as employed in context of the present invention is animal blood plasma or blood serum. Most preferably, the body fluid used in the present invention is human blood plasma or blood serum. The terms “blood plasma” and “blood serum” are well known in the art. In the context of the present invention the term “blood plasma” particularly means the liquid component of blood which may comprise proteins including fibrinogen, globulin and human serum albumin. A person skilled in the art may easily understand how to obtain blood plasma from whole blood which comprises blood cells including red blood cells, white blood cells and platelets. For example, plasma may be obtained by centrifugation of whole blood, thus removing blood cells. In the present invention, it is particularly envisaged that some blood cells are not removed from the plasma, for example by a first centrifugation step. This implies that blood cells or cell debris and the like may still be present in plasma samples to be selected in accordance with the present invention. The term “blood serum” particularly means in the context of the present invention blood plasma in which clotting factors, such as fibrin, have been removed. The person skilled in the art will be aware of methods for preparing blood serum, e.g. by removing clotting factors. One simple way to achieve this is by allowing the blood to clot prior to isolating the fluid, namely the serum.

In a preferred embodiment of the present invention, the DNA sample is derived from fresh blood plasma or fresh blood serum. However, due to the high performance of the disclosed method also DNA samples derived from frozen plasma or frozen blood serum can be selected. In the context of the present invention the term “frozen plasma” or “frozen serum” particularly means that the blood plasma or blood serum is frozen after collection and, optionally, stored in a frozen state. For example, said frozen plasma or frozen serum has been stored for at least 1 year, for at least 5 years or for at least 10 years. Furthermore, the plasma or serum may also have been dried before freezing or storage.

Any cell, tissue, or body fluid described herein and in the appended example that has been frozen and/or stored for at least 1, more preferably 5 and most preferably 10 years may be used according to the method of the present invention.

DNA samples derived from at least one cell or a tissue that has been preserved is also envisioned. Preservation methods are known to the artisan and include but are not limited to plastination, freeze-drying, vacuum drying and preservation methods comprising the use of glucose, glycerol, thymol, liquid nitrogen and phenol.

The “DNA sample” to be selected in context of this invention may, preferably, be a sample comprising extracted/isolated DNA, for example DNA isolated from organic material prior to the step of pre-genotyping or genotyping. However, it is pointed out that the DNA sample selected in accordance with the present invention may be a biological sample or a sample comprising (crude) organic material and hence, un-extracted/un-isolated DNA.

“DNA” as employed in the present invention is genomic DNA. The meaning of the term “genomic DNA” is well known in the art and may, for example, be deduced from Knippers (2006, Thieme Verlag). In the context of the present invention, the term “genomic DNA” particularly means DNA which is derived from a genome. Also the term “genome” is known to the artisan and may be deduced, for example, from Knippers (loc. cit.). In the context of the present invention, the term “genome”, for example, means the whole hereditary information which is encoded in DNA. “Genomic DNA” (“gDNA”) is envisaged to also encompass “genomic RNA” (“gRNA”) or “nucleolar RNA”, non-spliced RNA or partially spliced RNA. RNA samples may, inter alia, be transcribed to “DNA samples” by processes like “reverse transcription”. Such RNA/DNA samples may also be selected in accordance with the invention. It is to be understood that nucleic acid molecules may be modified to resemble DNA. For example, RNA may be transformed into DNA using, e.g. reverse transcriptase. Accordingly, nucleic acid molecules other than DNA may be assessed in genotyping and other molecular assessments when they are modified to resemble DNA. All definitions given herein in respect to “DNA samples”, “genomic DNA” apply to “nucleic acid samples” mutatis mutandis. Source for such RNA (to be reverse transcribed to DNA), resembling after modification (by e.g. reverse transcription) to a DNA sample or a “genomic DNA” as defined in accordance with this invention, comprise, but are not limited to viral RNA, spliced RNA, non-spliced RNA, partially spliced RNA and nucleolar RNA.

Preferably, the genomic DNA comprised in the DNA sample to be selected comprises the whole genomic DNA, for example the whole genomic DNA of the biological source/organism said DNA sample is derived from. However, it is also envisioned that genomic DNA comprises only (one) part(s) of a genome, for example one whole chromosome or several whole chromosomes, (one) part(s) of one chromosome or parts of several chromosomes. It is of note that generally no size exclusion limit exists with respect to the (genomic) DNA to be employed in accordance with the method of the present invention.

However, it is of note that it is intended in context of the present invention that the DNA sample to be selected most preferably represents the entire genome of a biological source/organism and hence, comprises the whole genomic DNA thereof.

Generally, the person skilled in the art is capable of extracting genomic DNA from a biological source/organism and is aware of corresponding (standard) techniques which can be deduced, for example, from Sambrook (2001, Cold Spring Harbour Laboratory Press).

For example, a first step of preparing an extract (of cells, a tissue or body fluid), for example a DNA extract, may comprise mechanical pulping, sonication, use of mortars and pestles, freeze-thawing cycles, use of blenders (like Waring-Blenders, Polytron), liquid homogenization and maceration or e.g. Dounce homogenization, Potter-Elvehjem, French Press etc. The technique chosen for the disruption of cells, whether physical or detergent-based, must take into consideration the origin of the cells or tissues being examined and the inherent ease or difficulty in disrupting their outer layer(s).

The freeze/thaw method is commonly used to lyse bacteria and cells from higher organism. The technique involves freezing a cell suspension in a dry ice/ethanol bath or freezer and then thawing the material at room temperature or 37° C. This method of lysis causes cells to swell and ultimately break as ice crystals form during the freezing process and then contract during thawing. Multiple cycles are necessary for efficient lysis, and the process can be quite lengthy.

Cells, organisms as well as tissue may be treated with various agents to aid the disruption process. Lysis can be promoted by suspending cells in a hypotonic buffer, which cause them to swell and burst more readily under physical shearing. Lysozyme can be used to digest the polysaccharide component of yeast and bacterial cell walls. Alternatively, processing can be expedited by treating cells with glass beads in order to facilitate the crushing of cell walls. Viscosity of a sample typically increases during lysis due to the release of nucleic acid material.

After disrupting the cell, tissue or organism, the released genomic DNA can be extracted by any of the methods known to a person skilled in the art. In particular, mechanical shearing of the genomic DNA should be avoided and special care should be taken in order to obtain a high yield of high-quality, non-degraded genomic DNA. Extraction of genomic DNA usually involves a salting out step, as used in the appended example and described in Miller (1988), Nucleic Acids Res 16, 1215. Alternatively, DNA extraction may be performed using CTAB (Cetyltrimethylammoniumbromid) in order to remove polysaccharides and proteins followed by dialysis. Proteins may also be removed by phenol-chloroform extraction which may be followed by ethanol or isopropanol precipitation of genomic DNA. The use of commercial kits or automated workstations for the extraction of genomic DNA is also envisioned. For example, the automated GenoM-48 Robotic workstation (GENOVISION, Qiagen) is used in the appended example.

In accordance with the method of the present invention, it is preferred that particularly a body fluid, such as blood plasma or blood serum, is centrifuged prior to cell disruption and extraction of genomic DNA. Preferably, the supernatant is discarded and the pellet is used for further processing, as shown in the appended example. The pellet may comprise cells, cell debris, nuclei or free genomic DNA. Less preferred, but also envisioned is the use of the supernatant for extraction of genomic DNA. Alternatively, the body fluid may not be centrifuged prior to extraction of genomic DNA, but directly subjected to cell lysis and DNA extraction.

A person skilled in the art will be capable of quantifying the concentration of (genomic) DNA comprised in the DNA sample by standard techniques which may be deduced, for example from Sambrook (2001), Cold Spring Harbour Laboratory Press; Hague (2003), BMC Biotechnol 3, 20. The concentration of the DNA solution may be quantified, for example, by UV-spectroscopy, PicoGreen® assay (Molecular Probes, Eugene, Oreg.) or Real time (RT) PCR, such as RT TaqMan assay specific to human DNA. In the appended example, genomic DNA was quantified by UV-absorbance measurement using the Nanoprop ND-1000 (Nanoprop Technologies, Wilmington, USA) and by Real time PCR using the Human Quantifiler Kit (Applied Biosystems, Germany) according to the Manufacturer's instructions. The concentration of the DNA solution may be quantified, for example, after the step of extraction of DNA.

The meaning of the term “genotyping” is well known to a person skilled in the art and may, for example, be deduced from Karl H. Hecker (2006; Genetic Variance Detection. DNA Press, LLC. Eagleville, Pa., USA). In the context of the present invention, “genotyping” particularly means a process of determining the genotype of an individual with a biological assay. Non-limiting examples for genotyping are Maldi-TOF genotyping, Taqman genotyping and microarray genotyping. It is preferred herein that genomic DNA as described above is employed in genotyping. Non-limiting examples for genotyping employed herein are single nucleotide polymorphism (SNP) genotyping, short tandem repeat (STR) genotyping, minisatellite genotyping and copy number variation genotyping. Particularly STR genotyping and SNP genotyping are described in the appended example. The corresponding definition given herein with respect to genotyping also applies with respect to the step of “pre-genotyping” performed according to the provided method, mutatis mutandis. However, preferably, pre-genotyping employed herein in accordance with the method of the present invention is STR genotyping. Alternatively, but less preferred, pre-genotyping may also be SNP genotyping. Non-limiting examples for SNP genotyping are Maldi-TOF SNP genotyping, Taqman SNP genotyping, SNP genotyping using chip technology. STR genotyping may, inter alia, be restriction fragment length polymorphism detection, sequencing and fragment analysis using PCR amplification

It is envisioned that a set of polymorphic markers used in the step of pre-genotyping may also be used in subsequent genotyping. However, it is of note that said set of polymorphic markers employed herein should not be used in association studies, for example disease association studies, if the markers are expected to be associated with the phenotype, in particular the disease, under investigation. Accordingly, In such a case a set of non-associated, polymorphic markers can be used.

Generally, the present invention provides for a method for selecting DNA samples which are (likewise) suitable for STR genotyping, SNP genotyping and other kinds of genotyping. The teaching provided herein implies that a suitable DNA sample selected in accordance with the present invention is of a sufficient quality for genotyping. Since the method provided herein allows for a general assessment of the quality of DNA samples it is generally envisaged that the DNA samples described herein are selected upon suitability for any kind of DNA analysis methods or genetic analyses. Non-limiting examples of such DNA analysis methods/genetic analyses are sequencing, genotyping and Southern Blots.

The meaning of the term “polymorphic marker” as used herein can, for example, be deduced from standard text books, like Encyclopedia of Genetics, Genomics, Proteomics and Bioinformatics, ISBN 9780470849743. In the context of the present invention, polymorphic markers particularly means that markers have a high heterozygosity rate, i.e. a high percentage of a distinct population has two alleles of said marker, while a low percentage of said population has only one out of said two alleles of said marker. The alleles differ, for example, with respect to SNP markers, in a single nucleotide or, with respect to STR markers, in the number of repeats of the respective marker. In a preferred embodiment of the present invention, the markers to be employed are highly polymorphic. Highly polymorphic markers employed herein have a heterozygosity rate of at least 70%, 75%, 80%, 85%, 90% or 95%. Particularly, highly polymorphic markers to be employed have a heterozygosity rate ranging from 70% to 95%, preferably from 71% to 94% more preferably from 72% to 94%. In a preferred embodiment of the invention, the polymorphic markers described herein are randomly distributed over the whole genome. More preferably, the polymorphic markers are evenly distributed over the whole genome. In case the genomic DNA comprises only parts of the whole genome, for example only several chromosomes or one chromosome or parts of at least one chromosome, the polymorphic markers employed in the method of the present invention should be present in said part of the whole genome. Preferably, the polymorphic primers are randomly distributed over said part of the whole genome. More preferably, the polymorphic primers are evenly distributed over said part of the whole genome.

A person skilled in the art knows that the phrase “(genomic) DNA homozygous for a marker” has the same meaning as the phrase “a homozygous marker” or “marker homozygous for (genomic) DNA”, and the like, and that these phrases can be used interchangeably. The corresponding definition given herein with respect to “genomic DNA homozygous for a marker”, “a homozygous marker” or “marker homozygous for (genomic) DNA” also applies with respect to “(genomic) DNA heterozygous for a marker”, “a heterozygous marker” or “marker heterozygous for (genomic) DNA”, and the like, mutatis mutandis.

“Set” of polymorphic markers as used herein generally means more than 1 polymorphic marker. Accordingly, the set of polymorphic markers employed herein comprises at least 2 polymorphic markers. Preferably, it may comprise at least 3, 4, 5, 6, 7, 8, 9, 10, 12, 15, 20, 25, 30 or 40 polymorphic markers. Most preferably, the set of polymorphic markers may comprise at least 9 polymorphic markers. A subgroup of the set of polymorphic markers defined herein above refers accordingly to a portion out of said set of polymorphic markers, wherein said portion/subgroup of polymorphic markers comprises at least 1 marker less than said set of polymorphic markers. For example, if the set of polymorphic markers comprises 9 markers, the subgroup may comprise 1, 2, 3, 4, 5, 6, 7, or 8 markers. If the set of polymorphic markers comprises only 2 markers, the subgroup thereof comprises 1 marker.

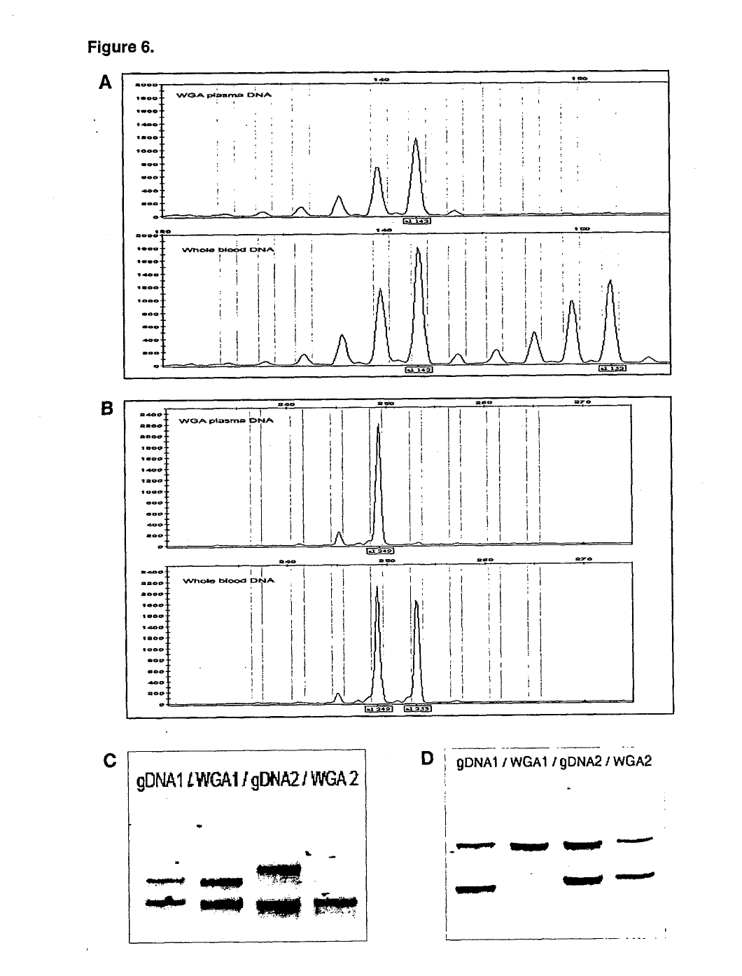

Within the method of the present invention, the step of pre-genotyping the genomic DNA as described herein is followed by a step of determining out of the set of polymorphic markers the percentage of polymorphic markers for which the genomic DNA is homozygous. A person skilled in the art will know how to determine the homozygosity of polymorphic markers and, accordingly, the percentage of polymorphic markers for which the DNA is homozygous. As exemplarily shown in the appended example, particularly in FIG. 6, genomic DNA which is homozygous for e.g. an STR marker may be characterized in that only one allele of the STR marker can be detected; in contrast, genomic DNA which is heterozygous for an STR marker may be characterized in that two alleles of the STR marker can be detected. A genomic DNA employed herein is considered homozygous for said marker if homozygosity is detected by pre-genotyping said genomic marker as described herein. A genomic DNA employed herein is considered heterozygous for said marker if heterozygosity is detected, mutatis mutandis. A person skilled in the art will be capable of calculating the percentage of homozygous markers out of the set of polymorphic markers for which the genomic DNA is homozygous by taking advantage of the teaching provided herein and his common general knowledge.

In the following, the two parameters that determine the probability of homozygosity of genomic DNA for polymorphic markers are described. The first parameter is the number of markers comprised in the set of polymorphic markers. The second parameter is the average heterozygosity rate of the markers comprised in the set of polymorphic markers. Both parameters determine the probability of homozygosity of genomic DNA for polymorphic markers. Thereby, one will find the following general correlation: the higher or lower the number of polymorphic markers, the lower or higher is the probability that a (genomic) DNA is homozygous for all of these markers. The higher or lower the average heterozygosity rate of the markers, the lower or higher is the probability that the genomic DNA is homozygous for all of the markers. In other words, the more polymorphic markers are used and the higher the average heterozygosity rate of said polymorphic markers is, the lower is the probability that a (genomic) DNA is homozygous for all of said polymorphic markers. In the context of the present invention it is preferred that the probability that the genomic DNA is homozygous for all of the markers/ a certain subgroup out of the set of polymorphic markers used is very low. Such a subgroup out of the set of polymorphic markers may comprise, for example, 5 markers out of a total number of 9 markers of the set of polymorphic markers. In a preferred embodiment of the invention, the probability for homozygosity of the genomic DNA for all markers/a certain subgroup out of the set of polymorphic markers is less than 2%. Preferably, the probability for homozygosity of the genomic DNA for all markers/ a certain subgroup out of the set of polymorphic markers is less than 1%. More preferably, the probability for homozygosity of the genomic DNA for all markers/ a certain subgroup out of the set of polymorphic markers is less than 0.9%, 0.8%, 0.7%, 0.6%, 0.5%, 0.4%, 0.3%, 0.2% or 0.1%. More preferably, the probability for homozygosity of the genomic DNA for all markers/ a certain subgroup out of the set of polymorphic markers is less than 0.09%, 0.08%, 0.07%, 0.06%, 0.05%, 0.04%, 0.03%, 0.02% or 0.01%. Even more preferably, the probability for homozygosity of the genomic DNA for all markers/ a certain subgroup out of the set of polymorphic markers is less than 0.009%, 0.008%, 0.007%, 0.006% or 0.005%. Most preferably, the probability for homozygosity of the genomic DNA for all markers/ a certain subgroup out of the set of polymorphic markers is less than 0.004%. Alternatively, the probability for homozygosity of the genomic DNA for all markers/ a certain subgroup out of the set of polymorphic markers is less than 0.03%, in particular when probability for homozygosity of the genomic DNA is calculated according to the formula II provided herein below. Though it is preferred that at least 9 markers are used, a different number of markers may be used in accordance with the method of the present invention, as long as the probability of homozygosity of the genomic DNA for the markers used is (far) below 2%, preferably below 1%, more preferably below 0.05%, more preferably below 0.04% and even more preferably below 0.03%.

A person skilled in the art knows how to calculate the probability of homozygosity of the genomic DNA for markers out of the set of polymorphic markers. An artisan will also be aware of the fact that the range of the heterozygosity rate of the polymorphic markers comprised in the set of polymorphic markers may influence the probability of homozygosity of genomic DNA for all markers/ a certain subgroup of the set of markers used. For example, the probability of homozygosity of a set of polymorphic markers which comprises markers each of which has a heterozygosity rate of e.g. 87 (86.8) % may differ from the probability of homozygosity of a set of polymorphic markers which comprises markers having a range of heterozygosity rates but an average heterozygosity rate of 87 (86.8) %. For example, the set of polymorphic markers shown in the appended example comprises 9 STR markers that have a heterozygosity rate of about 72% (e.g. for exemplified marker D12S2078 (SEQ ID No. 7)) and about 94% (e.g. for exemplified marker D5S2498 (SEQ ID No. 4)). The probability of homozygosity of all 9 markers of the set of markers used in the appended example is 0.000001216%, when the calculation according to formula Ia is based on the average heterozygosity rate. The probability of homozygosity of a certain subgroup of the set of polymorphic markers, for example 5 markers out of a total of 9 markers of the set of markers used in the appended example is 0.004007% (rounded off to 0.004%), when the calculation according to formula Ia (described herein below) is based on the average heterozygosity rate. The probability of homozygosity of a certain subgroup of the set of polymorphic markers, for example 5 markers out of a total of 9 markers of the set of markers used in the appended example is 0.027% (rounded off to 0.03%), when the calculation according to formula II (described herein below) is based on the average heterozygosity rate. In contrast, the probability of homozygosity of all 9 markers of the set of markers used in the appended example is 0.00000037735203210% when the calculation is based on the heterozygosity rates of the individual markers. Accordingly, the probability of homozygosity of a certain subgroup of the set of polymorphic markers, for example 5 markers (e.g. D1S495 (SEQ ID No. 1), D2S1338 (SEQ ID No. 2), D3S1314 (SEQ ID No. 3, D5S2498 (SEQ ID No. 4), D8S1130 (SEQ ID No. 5)) out of a total of 9 markers of the set of markers used in the appended example is 0.000783064792%, when the calculation is based on the heterozygosity rates of the individual markers.

As pointed out before, the low probability of homozygosity of the polymorphic markers described herein can likewise be obtained in the following cases:
1. Use of a relatively high number of polymorphic markers (e.g. more than 9 polymorphic markers) which have a relatively low average heterozygosity rate (e.g. less than 87%)
2. Use of a medium number of polymorphic markers (e.g. 9 polymorphic markers) which have a medium average heterozygosity rate (e.g. 87%)
3. Use of a relatively low number of polymorphic markers (e.g. less than 9 polymorphic markers) which have a relatively high average heterozygosity rate (e.g. more than 87%).

The relationship between the probability of homozygosity of genomic DNA and a given number of polymorphic markers having a given average heterozygosity rate can be described by the following formula Ia:

$$P ( 0 ) = {\prod }_{i = 0}^{m - 1} \frac{{n}_{0}}{{n}_{e} + {n}_{0}}$$

A more detailed formula Ib would be:

$$P ( 0 ) = {\prod }_{i = 0}^{m - 1} \frac{{n}_{0}}{{n}_{e} + {n}_{0} - m}$$

P(0): probability of homozygosity of (genomic) DNA (%)
m: number of markers
n₀: percentage of homozygous markers (%)
nₑ: percentage of heterozygous markers (%)

Number of markers m may be the total number of markers out of a set of (polymorphic) markers. The number of markers may also be the number of markers representing a subgroup of a set of (polymorphic) markers. Said number of markers representing a subgroup of a set of markers may, for example, be calculated by dividing the total number of markers out of the set of markers by 2 and rounding the resulting number up to the next natural number. This calculation refers to the particular situation when 5 markers out of a total number of 9 markers represent said subgroup of a set of (polymorphic) markers, for example 55.5% of said set of polymorphic markers. A person skilled in the art may easily adapt said calculation to different values of percentage. Similarly, one may calculate said number of markers representing a subgroup of a set of markers using the percentage of polymorphic markers out of said set of markers for which the DNA is homozygous for. The definitions given herein with respect to a set of (polymorphic) markers or a subgroup of (polymorphic) markers apply here, mutatis mutandis.

A person skilled in the art will know how to calculate the probability of homozygosity of genomic DNA P(0) by, for example, taking advantage of the above formula and the teaching provided herein. An artisan will also know how to calculate the number of markers m and/or the percentage of homozygous markers n₀, respectively the percentage of heterozygous markers nₑ, for a given probability of homozygosity of genomic DNA P(0) by, for example, taking advantage of the above formula.

In particular and in even more preferred embodiments of this invention, the relationship between the probability of homozygosity of genomic DNA and a given number of polymorphic markers having a given average heterozygosity rate can be described by the following formula II:

The cut-off value for the number of acceptable homozygous loci can be calculated using the probability mass function of the binomial distribution.

Let n be the number of markers comprised in the set of polymorphic markers as defined herein above and let q be the average heterozygosity rate of the different markers. Then, the average homozygosity rate p of the can be calculated as: p=1−q.

The probability, that a sample is homozygous in k out of n loci can then be calculated as:

$$P ( X = k ) = ( \begin{matrix}n \\ k\end{matrix} ) \cdot {p}^{k} \cdot {q}^{n - k}$$

### Procedure:

1) Calculate the average heterozygosity rate of your n loci
2) Set i=1
3) Set k=i
4) Calculate the probability P(X=k).
5) If P(X=k)>0.01 then set k=i+1 and go to step 4.
6) If P(X=k)≦0.01 (equivalent to 1%), then k is the cut-off number of homozygous loci, starting from which a sample may be selected for further analysis.

As described herein below, it is preferred that the probability for homozygosity of the genomic DNA for all markers out of the set of polymorphic markers or a certain subgroup out of the set of polymorphic markers is less than 2%, corresponding to an error probability of less than 2%, when said probability is determined according the above formula II. Preferably, said probability is below 1%, corresponding to an error probability of less than 1%, more preferably, below 0.9%, 0.8%, 0.7%, 0.6%, 0.5%, 0.4% or 0.3%, corresponding to an error probability of less than 0.9%, 0.8%, 0.7%, 0.6%, 0.5%, 0.4% or 0.3%, respectively. It is also envisaged though less preferred that the probability for homozygosity of the genomic DNA for all markers out of the set of polymorphic markers or a certain subgroup out of the set of polymorphic markers is less than 5%, corresponding to an error probability of less than 5%.

The following, non-limiting examples of the application of the above formulae may be given:

### A:

Imagine an STR-multiplex with 10 independent STR loci and an average heterozygosity rate of 85%.

$$n = 10$$

$$q = 0.85$$

$$p = 0.15$$

$$i = 1 \text{:} P ( X = 1 ) = ( \begin{matrix}10 \\ 1\end{matrix} ) \cdot {0.15}^{1} \cdot {0.85}^{9} = 0.3474 > 0.01 -> next i$$

$$i = 2 \text{:} P ( X = 2 ) = ( \begin{matrix}10 \\ 2\end{matrix} ) \cdot {0.15}^{2} \cdot {0.85}^{8} = 0.2759 > 0.01 -> next i$$

$$i = 3 \text{:} P ( X = 3 ) = ( \begin{matrix}10 \\ 3\end{matrix} ) \cdot {0.15}^{3} \cdot {0.85}^{7} = 0.1298 > 0.01 -> next i$$

$$i = 4 \text{:} P ( X = 4 ) = ( \begin{matrix}10 \\ 4\end{matrix} ) \cdot {0.15}^{4} \cdot {0.85}^{6} = 0.0401 > 0.01 -> next i$$

$$i = 5 \text{:} P ( X = 5 ) = ( \begin{matrix}10 \\ 5\end{matrix} ) \cdot {0.15}^{5} \cdot {0.85}^{5} = 0.0085 \leq 0.01 -> At this step , the probability for homozygosity of the genomic D N A for five markers ( which can be considered as a certain subgroup of the set of \\ markers ) \text{/} error probability is below 1 \% . As described above , the probability for homozygosity of the genomic D N A for all markers out of the set of polymorphic markers or a certain subgroup out of the set of polymorphic markers \text{/} error probability is preferably less than 1 \% . However , as described herein above , the probability for homozygosity \text{/} error probability may also be , for example , less than 5 \% or less than 2 \% , wherein the lower value is preferred .$$

Thus, a sample pre-genotyped in accordance with the present application and found to be homozygous for, for example, less than 50% (5 out of 10 markers; see the above example A) of the exemplary set of polymorphic markers can be selected for further analysis. In other words, as soon as the probability for homozygosity of genomic DNA for the set of markers or a subgroup thereof falls below, for example, 1%, a sample pre-genotyped in accordance with the present application and found to be homozygous for, for example, less than 50% (5 out of 10 markers) of the exemplary set of polymorphic markers will be selected for further molecular analysis/assessment, such as genotyping or other molecular assessments.

Further examples of the application of the above formulae given herein below may illustrate the relationship between the probability of homozygosity of genomic DNA for the set of markers or a subgroup thereof, the average heterozygosity rate of the markers, the number of markers, and the percentage of homozygous markers which determines whether a DNA sample is selected in accordance with the present invention.

### B.

Imagine an STR-multiplex with 10 independent STR loci and an average heterozygosity rate of 70%. The upper limit of the probability of a genomic DNA for homozygosity for a set of polymorphic markers (error probability) is set to 1%.

$$P < 0.01$$

$$n = 10$$

$$q = 0.70$$

$$p = 0.30$$

$$i = 1 \text{:} P ( X = 1 ) = ( \begin{matrix}10 \\ 1\end{matrix} ) \cdot {0.3}^{1} \cdot {0.7}^{9} = 0.1211 > 0.01 -> next i$$

$$i = 2 \text{:} P ( X = 2 ) = ( \begin{matrix}10 \\ 2\end{matrix} ) \cdot {0.3}^{2} \cdot {0.7}^{8} = 0.2335 > 0.01 -> next i$$

$$i = 3 \text{:} P ( X = 3 ) = ( \begin{matrix}10 \\ 3\end{matrix} ) \cdot {0.3}^{3} \cdot {0.7}^{7} = 0.2668 > 0.01 -> next i$$

$$i = 4 \text{:} P ( X = 4 ) = ( \begin{matrix}10 \\ 4\end{matrix} ) \cdot {0.3}^{4} \cdot {0.7}^{6} = 0.2001 > 0.01 -> next i$$

$$i = 5 \text{:} P ( X = 5 ) = ( \begin{matrix}10 \\ 5\end{matrix} ) \cdot {0.3}^{5} \cdot {0.7}^{5} = 0.1029 > 0.01 -> next i$$

$$i = 6 \text{:} P ( X = 6 ) = ( \begin{matrix}10 \\ 6\end{matrix} ) \cdot {0.3}^{6} \cdot {0.7}^{4} = 0.0367 > 0.01 -> next i$$

$$i = 7 \text{:} P ( X = 7 ) = ( \begin{matrix}10 \\ 7\end{matrix} ) \cdot {0.3}^{7} \cdot {0.7}^{3} = 0.0090 \leq 0.01 -> If a sample shows at least 7 homozygous loci , then is excluded from further analysis . In other words , if a sample is found to be homozygous for less than 70 \% of the set of polymorphic markers ( 7 markers out of 10 markers ) after the step of pre \text{-} genotyping , said sample is selected for further analysis ..$$

### C.

Imagine an STR-multiplex with 8 independent STR loci and an average heterozygosity rate of 87%. The upper limit of the probability of a genomic DNA for homozygosity for a set of polymorphic markers (error probability) is set to 5%.

$$P < 0.05$$

$$n = 8$$

$$q = 0.87$$

$$p = 0.13$$

$$i = 1 \text{:} P ( X = 1 ) = ( \begin{matrix}8 \\ 1\end{matrix} ) \cdot {0.13}^{1} \cdot {0.87}^{7} = 0.3924 > 0.01 -> next i$$

$$i = 2 \text{:} P ( X = 2 ) = ( \begin{matrix}8 \\ 2\end{matrix} ) \cdot {0.13}^{2} \cdot {0.87}^{6} = 0.2052 > 0.01 -> next i$$

$$i = 3 \text{:} P ( X = 3 ) = ( \begin{matrix}8 \\ 3\end{matrix} ) \cdot {0.13}^{3} \cdot {0.87}^{5} = 0.0613 > 0.01 -> next i$$

$$i = 4 \text{:} P ( X = 4 ) = ( \begin{matrix}8 \\ 4\end{matrix} ) \cdot {0.13}^{4} \cdot {0.87}^{4} = 0.0115 < 0.05 -> If a samples shows at least 4 homozygous loci , then is excluded from further analysis . Accordingly , a sample is selected for further analysis , if said sample is , after the step of pre \text{-} genotyping , found to be homozygous for less than 40 \% ( 4 markers out of 8 markers ) of the set of polymorphic markers .$$

The following tables summarize the results of exemplary, non-limiting uses of the above formulae In context of this invention illustrating the relationship between the probability of homozygosity of genomic DNA for the set of markers or a subgroup thereof, the average heterozygosity rate of the markers, the number of markers, and the percentage of homozygous markers which determines whether a DNA sample is selected in accordance with the present invention.

D. Probability of a genomic DNA for homozygosity for a set of polymorphic markers or a subgroup thereof/error probability of less than 5%:

| | Average heterozygosity rate | | |
|---|---|---|---|
| | 70% | 80% | 83% |
| Number of | 10 | 8 | 8 |
| STRs/markers to | | | |
| type | | | |
| Percentage of | Less than 60% | Less than 50.0% | Less than 50.0% |
| homozygous | (Less than 6 out of | (Less than 4 markers | (Less than 4 out of 8 |
| STRs/markers, below | 10 markers | out of 8 | markers |
| which a sample can | homozygous) | homozygous) | homozygous) |
| be selected for | | | |
| further genotyping | | | |

| | Average heterozygosity rate | | | |
|---|---|---|---|---|
| | 85% | 87% | 90% | 95% |
| Number of | 8 | 8 | 6 | 5 |
| STRs/markers to | | | | |
| type | | | | |
| Percentage of | Less than 50.0% | Less than 50.0% | Less than 50.0% | Less than 40.0% |
| homozygous | (Less than 4 out | (Less than 4 out | (Less than 3 out | (Less than 2 |
| STRs/markers, | of 8 markers | of 8 markers | of 6 markers | markers out of 5 |
| below which a | homozygous) | homozygous) | homozygous) | homozygous) |
| sample can be | | | | |
| selected for | | | | |
| further | | | | |
| genotyping | | | | |

D. Probability of a genomic DNA for homozygosity for a set of polymorphic markers or a subgroup thereof/error probability of less than 1%:

| | Average heterozygosity | | | |
|---|---|---|---|---|
| | 70% | 80% | 83% | 85% |
| Number of | 10 | 8 | 8 | 8 |
| STRs/markers to | | | | |
| type | | | | |
| Percentage of | Less than | Less than | Less than | Less than |
| homozygous | 70.0% | 62.5% | 62.5% | 62.5% |
| STRs/markers, | (Less than 7 out | (Less than 5 out | (Less than 5 out | (Less than 5 out |
| below which a | of 10 markers | of 8 markers | of 8 markers | of 8 markers |
| sample can be | homozygous) | homozygous) | homozygous) | homozygous) |
| selected for | | | | |
| further | | | | |
| genotyping | | | | |

| | Average heterozygosity rate | | |
|---|---|---|---|
| | 87% | 90% | 95% |
| Number of | 8 | 6 | 5 |
| STRs/markers to type | | | |
| Percentage of | Less than 62.5% | Less than 66.7% | Less than 60.0% |
| homozygous | (Less than 5 out of 8 | (Less than 4 out of 6 | (Less than 3 out of 5 |
| STRs/markers, below | markers homozygous) | markers homozygous) | markers homozygous) |
| which a sample can be | | | |
| selected for further | | | |
| genotyping | | | |

The use of the above formulae has a wide dynamic range in terms of the average heterozygosity rate: if the average heterozygosity rate lies between 80-90%, it will be sufficient to type 8 STRs to select a sample, if less than 4 (for a 5% probability of a genomic DNA for homozygosity for a set of polymorphic markers or a subgroup thereof/error probability) or less than 5 (for a 1% probability of a genomic DNA for homozygosity for a set of polymorphic markers or a subgroup thereof/error probability) markers are homozygous.

Generally, for a given probability (e.g. below 1° A) or below 5%) of homozygosity of genomic DNA for the set of markers or a subgroup thereof either a higher number of markers (e.g. 10) with a lower average heterozygosity rate (e.g. 70%), an intermediate number of markers (e.g. 8) with an intermediate average heterozygosity rate (e.g. 80% to 87%), or a lower number of markers (e.g. 6) with a higher average heterozygosity rate (e.g. 90%) may be used in accordance with the present method. Of course, use of different sets of polymorphic markers will result in different specific probabilities of homozygosity of genomic DNA for the set of markers or a subgroup thereof. This means that the probability for one specific set of markers (or subgroup thereof) will be below e.g. 1% if, for example, less than 7 out of 10 markers are homozygous. For another set of markers the probability (or subgroup thereof) will be below e.g. 1% if, for example, less than 5 out of 8 markers are homozygous.

Thus, a different set of markers to be used in accordance with the present invention may result in different percentages of homozygous markers determining whether a DNA sample is selected.

The following text passage explains in more detail the relationship between the probability of homozygosity of genomic DNA for the set of markers or a subgroup thereof, the average heterozygosity rate of the markers, the number of markers, and the percentage of homozygous markers which determines whether a DNA sample is selected in accordance with the present invention.

The higher the probability that a genomic DNA is in a real state heterozygous for a set of markers or a subgroup thereof, the lower is the probability that a genomic DNA is in a real state homozygous for a set of markers or a subgroup thereof and vice versa.

The lower the probability that a genomic DNA is in a real state homozygous for a set of markers or a subgroup thereof, the higher is the probability that a genomic DNA which appears to be homozygous for a set of markers (or a subgroup thereof), e.g. after a step of pre-genotyping as described herein, is in a real state not homozygous for said set of markers or a subgroup thereof.

The higher the probability that a genomic DNA is not homozygous (though it may appear to be homozygous due to, for example, pre-genotyping), the higher is the probability that the result obtained by, for example, pre-genotyping, in respect of homozygosity of a genomic DNA for a set of markers (or a subgroup thereof) does not represent the real state of homozygosity of the genomic DNA. In other words, the probability that the result of pre-genotyping in respect of homozygosity of the genomic DNA is false-positive, is higher.

The higher the probability of false-positive results in respect of homozygosity of the genomic DNA, for example, in pre-genotyping is, the higher is the probability that the quality of the DNA is low and accordingly, the probability that a further analysis (e.g. genotyping) of said DNA will give false results is higher.

In sum, one can say that the lower the probability is that a genomic DNA is in a real state homozygous for a set of markers (or a subgroup thereof) the lower is the probability that results obtained for example by pre-genotyping which show an apparent homozygosity of genomic DNA for a set of markers (or a subgroup thereof) represent the real state of homozygosity for a set of markers (or a subgroup thereof)

Therefore, low values of the probability of homozygosity of genomic DNA for a set of markers or a subgroup thereof are preferred, as explained also in detail herein below. In the end, the probability of homozygosity of genomic DNA for a set of markers or a subgroup thereof as defined herein above influences the percentage of homozygous markers which determines whether a DNA sample is selected. The lower said percentage of homozygous markers is, the higher is the quality of the DNA sample selected in accordance with the present method and the more reliable are the results obtained in further analysis (e.g. genotyping) of the DNA sample. The higher quality of the DNA sample is reflected in a decrease in the discordance rate as described herein and shown in the appended example. However, a low percentage of homozygous markers in the selection step of the present method will result in a lower percentage of samples selected out of all samples pre-genotyped in accordance with the present invention. Preferably, less than 40%, 35%, 30% and most preferably, less than 25% of the samples pre-genotyped according to the method of the invention are excluded from further analysis.

The above describes the relationship between the probability of homozygosity of genomic DNA for the set of markers (or a subgroup thereof), the average heterozygosity rate of the markers, the number of markers, and the percentage of homozygous markers below which a DNA sample is selected. In the following individual parameters are described.

Generally, the average heterozygosity rate of the set of polymorphic markers employed herein may also be low, e.g. 30%, 10% or even below 1%.

However, it is preferred that the average heterozygosity rate of the set of polymorphic markers is high, for example at least 70%. Otherwise a high number of polymorphic markers has to be used in order to obtain a comparable low probability of homozygosity of DNA for a certain set/subgroup of polymorphic markers described herein above. For example, the use of a certain set of polymorphic markers which have a low average heterozygosity rate will increase the probability of homozygosity of DNA samples for this set of polymorphic markers, or a subgroup thereof, compared to a set of polymorphic markers having the same amount of markers but with a higher average heterozygosity rate. Thus, it may be advantageous to validate the set of polymorphic markers to be employed by determining the discordance rate of DNA sample pairs, one sample having high quality DNA and the other sample having low DNA quality. A non-limiting example for a sample having high quality DNA may be a sample derived from whole blood while, for example, a sample derived from blood plasma may comprise low quality DNA. An example for such a validation as described above is also shown in the appended example. In context of such a validation, one may expect, that more samples may have to be excluded in such a situation (lower boundary in the number of homozygote markers) to keep the rate of discordances low between the whole blood and plasma DNA samples.

The polymorphic markers comprised in the set of polymorphic markers described and defined herein and employed in accordance with the present invention may have an average heterozygosity rate of at least 65%, 70%, 75%, 80%, 85%, 86% or at least 87 (86.8) %. Thereby, the higher values of percentage are preferred. A non-limiting example for a highly polymorphic marker is the Kringle-IV repeat polymorphism in the LPA gene which has a heterozygosity rate of about 93%. Exemplary highly polymorphic markers to be used in accordance with the present invention are described herein below and in the appended Example. However, a person skilled in the art is easily in the position to identify and to select highly polymorphic markers that may be used in context of the present invention. These polymorphic markers are, for example disclosed in the following databases and may be deduced therefrom: National Center for Biotechnology (http://www.ncbi.nlm.nih.gov), The GDB Human Genome Database (http://www.gdb.org) and Cooperative Human Linkage Center (http://gai.nci.nih.gov/CHLC). In addition, markers to be used in the present method are described in commercially available test kits, such as AmpFlSTR® SGM Plus® from Applied Biosystems for detection of 10 STRs and the gender-determining locus Amelogenin, AmpFlSTR® Profiler Plus™ from Applied Biosystems for detection of 10 STRs and the Amelogenin locus, AmpFlSTR® COfiler™ for detection of 13 STRs, PowerPlex® 16 kit from Promega Corporation for detection of 15 STRs and the Amelogenin locus, Identifiler™ from Applied Biosystems for detection of 15 STRs and the Amelogenin locus, AmpFlSTR® SEfiler™ from Applied Biosystems for detection of 11 STRs and the Amelogenin locus. A skilled person will be aware of further kits the markers described therein can be used in the present invention. Generally and as described herein above, any marker can be used in context of the present invention as long as it has an average heterozygosity rate of at least 70%.

Non-limiting examples of highly polymorphic markers (Heterozygosity rate observed in Caucasians of the individual marker indicated in brackets) can be used in context of the present invention are: D1S1656 (89.2%), D8S1132 (84.8%), D10S2325 (85.6%), D3S1358 (80.1%), vWA (81.1%), TH01 (82.0%), D7S820 (81.8%), D2S1338 (89.6%), D8S1179 (81.1%), D21S11 (85.0%), D18S51 (86.4%) and/or FGA (85.2%). These and other highly polymorphic markers to be used in the present selection method are well known in the art. For example, Italian population data for the highly polymorphic markers D1S1656, D3S1358, D8S1132, D10S2325, VWA, FES/FPS, and F13A01 is described in De Leo D, Turrina S, Marigo M, Tiso N, Danieli G A (2001). Forensic Sci Int 123(1):71-73. Allele frequencies for 12 autosomal short tandem repeat loci in two Bolivian populations is described in Cifuentes L, Jorquera H, Acuña M, Ordóñez J, Sierra A L (2008). Highly polymorphic markers to be used in the present method are also described in Genet Mol Res 7(1):271-275) and in the above mentioned commercially available kits.

In one specific embodiment of the present invention, the set of polymorphic markers to be employed is envisaged to comprise at least 5 polymorphic markers and the average heterozygosity rate of said 5 polymorphic markers is envisaged to be at least 87 (86.8) %. In one particular example of this embodiment, the set of polymorphic markers to be employed comprises 9 polymorphic markers having an average heterozygosity rate of at least 87 (86.8) %.

As mentioned above, a DNA sample is selected in the context of the present invention when its DNA is homozygous for less than 60% of the set of polymorphic markers employed. In other words, a DNA sample according to the teaching of the present invention is suitable for genotyping when its DNA is homozygous for less than 60% of the markers from the employed set of polymorphic markers.

As demonstrated in the appended example, the selection of such DNA samples which DNA is homozygous for less than 60% of the employed polymorphic markers ensures (at a high probability) that said DNA has high quality, i.e. a quality sufficient for save genotyping approaches.

In one specific embodiment, the threshold value when a DNA sample is selected according to the method of the present invention is below 60% homozygosity for the employed set of polymorphic markers. Examples of such lower threshold values are 55.5%, 50%, 45%, 44.4%, 40%, 35%, 33.3%, 30% or even lower values. As described above, the probability of homozygosity of a DNA sample employed herein for a given set of (highly) polymorphic markers or a subgroup thereof is far below 2%. This means in other words that the probability that the apparent homozygosity of a DNA sample for a given set of (highly) polymorphic markers (or a subgroup thereof) represents the real state of the DNA sample is far below 2%. Vice versa, the probability that the DNA sample is in reality heterozygous for at least one polymorphic marker out of said set of polymorphic markers (or said subgroup thereof) is far above 98%. It is known in the prior art that extreme dilution of DNA favours genotyping errors such as allelic dropouts, see Pompanon (2005; loc. cit.). Thus, a DNA sample which is in reality heterozygous for a polymorphic marker may appear to be homozygous due to said genotyping error. Taken together, the percentage of homozygosity of a DNA sample for a given set of polymorphic markers (or a subgroup thereof) as described herein is a measure for the quality of said DNA sample. For example, a DNA sample having a high percentage of homozygosity for the set of polymorphic markers most likely comprises low quality DNA and is thus not suitable for genotyping. In other words, excluding such DNA samples having a high percentage of homozygosity for the set of polymorphic markers employed herein or, respectively, selecting such DNA samples having a low percentage of homozygosity for the set of polymorphic markers, in particular less than 70%, 69%, 68%, 67%, 66%, 65%, 64%, 63%, 62%, 61% or 60%. Also, as described in the appended examples selecting DNA samples having a percentage of homozygosity for the set of polymorphic markers of less than 60%, 56%, 55.5%, 50%, 45%, 44.5%, 40%, 35% or 33.3%, will dramatically improve genotyping results. This has, for example, been shown in the appended example. For example, the exclusion of 22.7% of the DNA samples resulted in a dramatic 4-fold decrease in the discordance for STR genotypes and an even more pronounced 6.5-fold decrease in the discordance for SNP genotypes.

It is evident from the above said and the appended example that the increase in the improvement of genotyping results will be the higher the lower the percentage of homozygosity for the set of polymorphic markers of selected DNA samples according to the method disclosed herein is. On the one hand, it is desirable that the genotyping results are highly improved. However, this might lead to the exclusion of all DNA samples but one, in an extreme example. On the other hand, as many samples as possible should be selected in order to avoid loss of genotyping data. Thus, it is evident that there is a trade-off between accuracy or improvement of genotyping results and loss of samples due to exclusion, a correlation also the data of the appended example point to.

For example, if one performs a case-control study and expects a large effect size of a given polymorphism on the phenotype, particularly the disease, the number of exclusions can be kept smaller by excluding only DNA samples with a higher number of homozygotes. If the expected effect size is small, an association can easily be disturbed in case of an increased frequency of allelic drop outs. Therefore, already samples with a medium number of homozygotes have to be excluded. Furthermore, such a study should only be applied, if in cases and controls the same genotype distribution of the (STR) markers which are used for the selection of reliable samples, can be expected. That means that these (STR) markers should not be related to phenotype investigated in the case-control study. Thus, it is clear for a person skilled in the art that the choice of a set of markers having a particular average heterozygosity rate depends on the chosen approach.

However, all of these exemplified specific genotyping approaches can be improved by pre-selecting the DNA samples to be employed according to the method of this invention.

The set of polymorphic markers may, inter alia, be a set of short tandem repeat markers (STR) or single nucleotide polymorphism (SNP) markers. However, a set of SNP markers is less preferred, since the heterozygosity rate of SNP markers is usually lower than that of STR markers. The usually higher rate of heterozygosity makes STR markers also more sensitive for allelic dropouts compared to SNP markers. Accordingly, a set of STR markers is preferred. Also a set of different kind of polymorphic markers, e.g. a mixed set of SNP and STR markers can be employed.

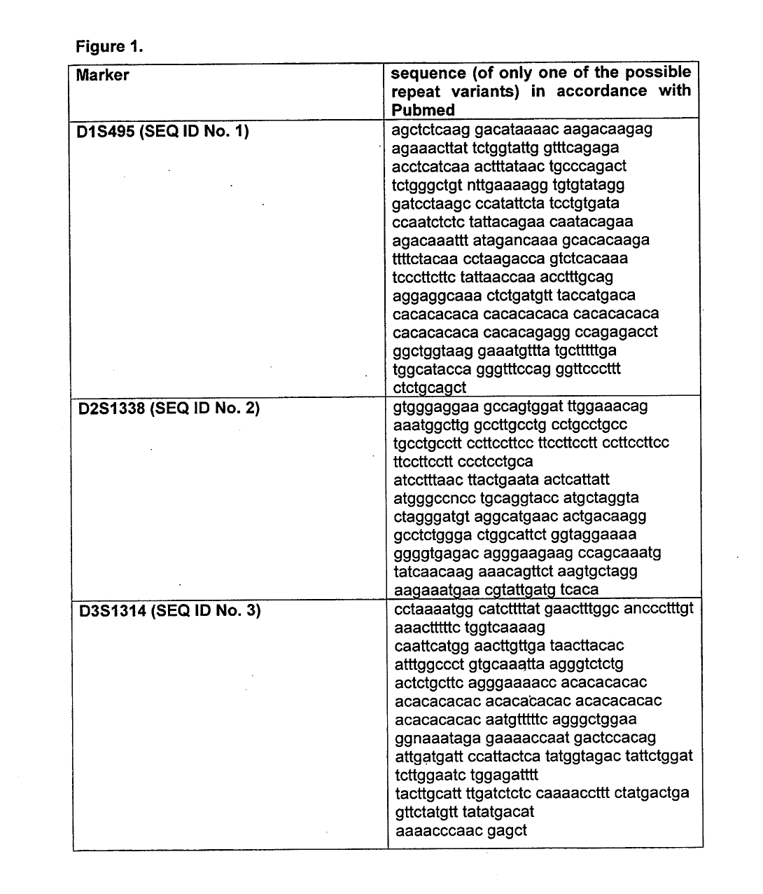

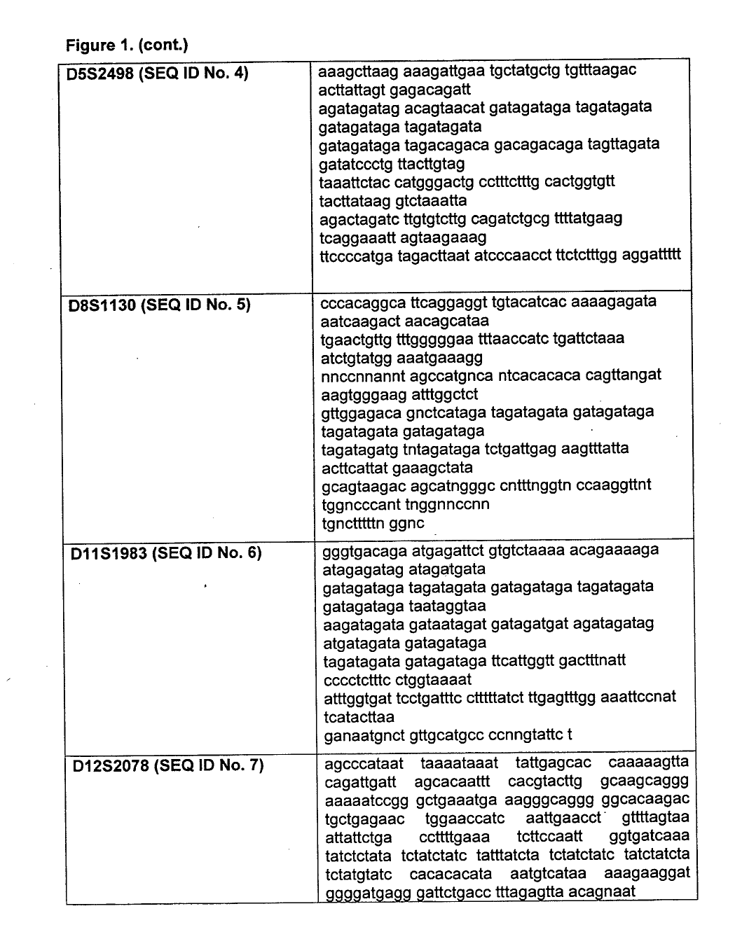

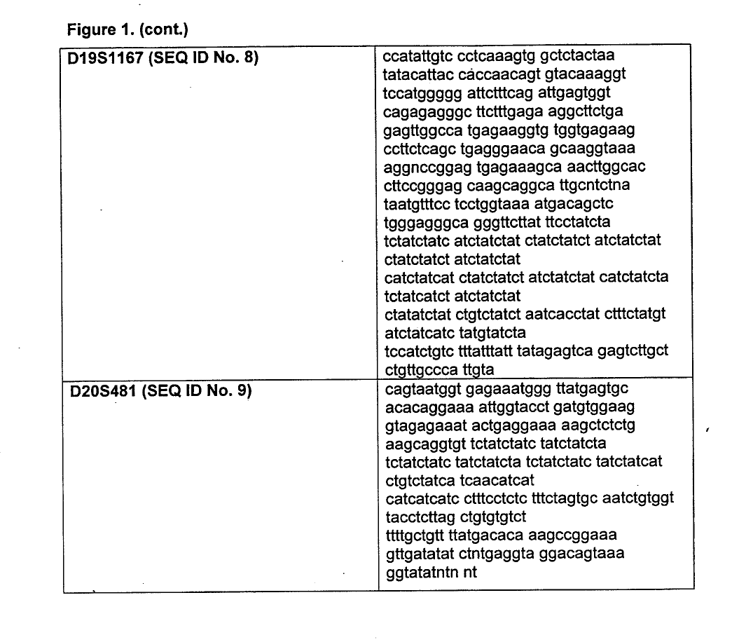

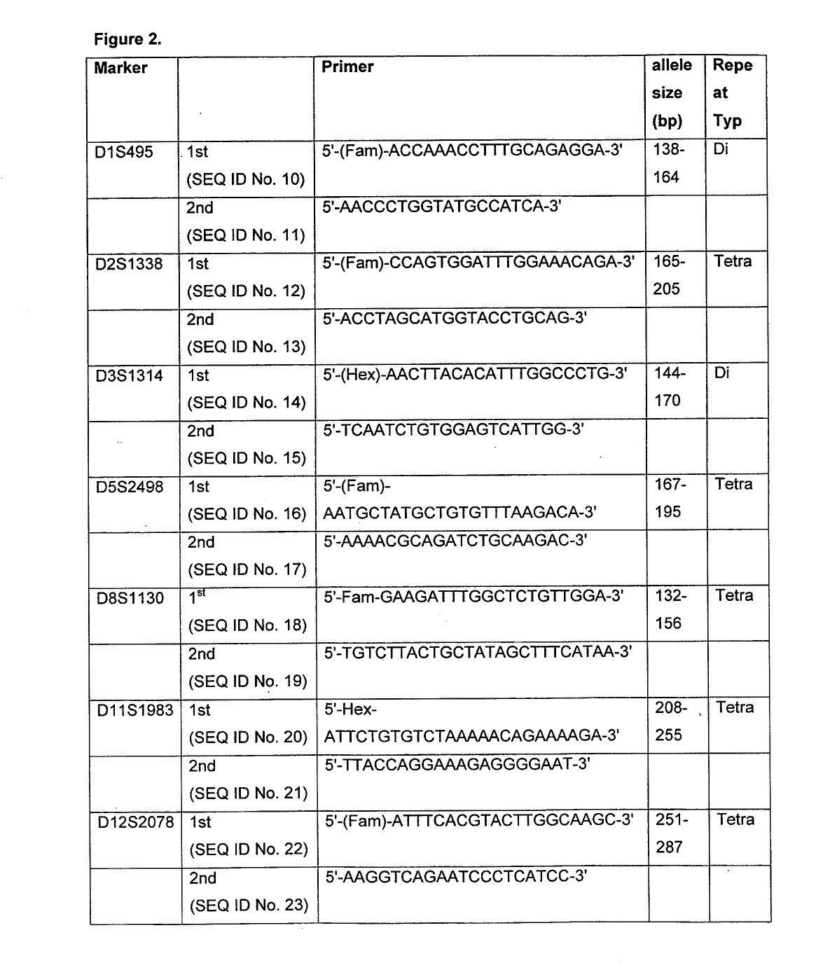

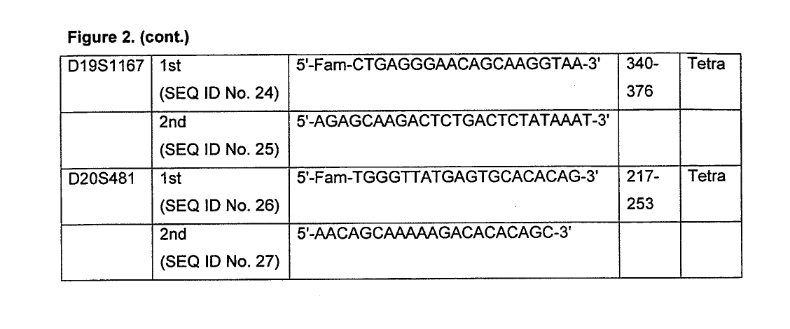

One particular set of highly polymorphic markers to be employed in context of the herein provided method is described herein and in the appended example. Particularly, the set of polymorphic markers employed herein may, inter alia, comprise one or more polymorphic marker selected from the group consisting of D1S495 (SEQ ID No. 1), D2S1338 (SEQ ID No. 2), D3S1314 (SEQ ID No. 3), D5S2498 (SEQ ID No. 4), D8S1130 (SEQ ID No. 5), D11S1983 (SEQ ID No. 6), D12S2078 (SEQ ID No. 7), D19S1167 (SEQ ID No. 8), and D20S481 (SEQ ID No. 9). Said 9 highly polymorphic STR markers, also described in FIG. 1, have been used in the particular marker set of the appended example. These exemplarily disclosed STR markers are distributed over 9 different chromosomes, have a heterozygosity rate ranging from about 72% to about 94%, mean 87 (86.8)±7% and may, inter alia, be employed for STR genotyping in accordance with the method of the present invention. These particular markers have an individual heterozygosity rate of 90.9% (D1S495; SEQ ID No. 1), 85.1% (D2S1338; SEQ ID No. 2), 93.2% (D3S1314; SEQ ID No. 3), 94.3% (D5S2498; SEQ ID No. 4), 85.1% (D8S1130; SEQ ID No. 5), 92.0% (D11S1983; SEQ ID No. 6), 71.6% (D12S2078; SEQ ID No. 7), 89.5% (D19S1167; SEQ ID No. 8), and 79.8% (D20S481; SEQ ID No. 9). The markers D1S495, D2S1338, D3S1314, D5S2498, D8S1130, D11S1983, D12S2078, D19S1167, D20S481 are disclosed in the following databases: National Center for Biotechnology (http://www.ncbi.nlm.nih.gov), The GDB Human Genome Database (http://www.gdb.org) and Cooperative Human Linkage Center (http://gai.nci.nih.gov/CHLC). The following primers, also shown in FIG. 2, have been used for STR genotyping of the respective markers in the appended example and may, inter alia, be used for the pre-genotyping in accordance with the method provided herein: D1S495 (first primer, SEQ ID No. 10) for detecting a Di repeat type of said STR marker; D1S495 (second primer, SEQ ID No. 11) for detecting a Di repeat type of said STR marker; D2S1338 (first primer, SEQ ID No. 12) for detecting a Tetra repeat type of said STR marker; D2S1338 (second primer, SEQ ID No. 13) for detecting a Tetra repeat type of said STR marker; D3S1314 (first primer, SEQ ID No. 14) for detecting a Di repeat type of said STR marker; D3S1314 (second primer, SEQ ID No. 15) for detecting a Di repeat type of said STR marker; D5S2498 (first primer, SEQ ID No. 16) for detecting a Tetra repeat type of said STR marker; D5S2498 (second primer, SEQ ID No. 17) for detecting a Tetra repeat type of said STR marker; D8S1130 (first primer, SEQ ID No. 18) for detecting a Tetra repeat type of said STR marker; D8S1130 (second primer, SEQ ID No. 19) for detecting a Tetra repeat type of said STR marker; D11S1983 (first primer, SEQ ID No. 20) for detecting a Tetra repeat type of said STR marker; D11S1983 (second primer, SEQ ID No. 21) for detecting a Tetra repeat type of said STR marker; D12S2078 (first primer, SEQ ID No. 22) for detecting a Tetra repeat type of said STR marker; D12S2078 (second primer, SEQ ID No. 23) for detecting a Tetra repeat type of said STR marker; D19S1167 (first primer, SEQ ID No. 24) for detecting a Tetra repeat type of said STR marker; D19S1167 (second primer, SEQ ID No. 25) for detecting a Tetra repeat type of said STR marker; D20S481 (first primer, SEQ ID No. 26) for detecting a Tetra repeat type of said STR marker; D20S4815 (second primer, SEQ ID No. 27) for detecting a Tetra repeat type of said STR marker. These primers are, e.g., disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

In another particular and preferred embodiment of the invention, the method disclosed herein further comprises the step of amplifying genomic DNA from the DNA sample prior to the step of pre-genotyping. In general, the skilled person is capable of amplifying genomic DNA from a cell, a tissue or body fluid by standard techniques which can be deduced, for example, from Sambrook (2001), Cold Spring Harbour Laboratory Press. Any method comprising the use of a DNA polymerase suitable for the amplification of DNA may be used. For example, polymerase chain reaction can be used for amplification of (genomic) DNA. Primers used in the amplification process may comprise oligonucleotides or hexamers. Primers used may be suitable for the amplification of specific parts of the genome, for example one or more chromosomes, parts of one or more choromosomes or stretches comprising at least one locus on the genome. It should be pointed out that any part of the genome may be amplified independent of its sequence or length as long as the amplified genomic DNA allows for pre-genotyping or genotyping. The use of specific primers is envisaged but less preferred in the amplification of (genomic) DNA. Preferably, random primers are used for amplifying (genomic) DNA in context of the invention.

In a preferred embodiment of the method of the present invention whole genome amplification (WGA) is used for amplifying genomic DNA. Most preferably, the step of amplifying genomic DNA comprises multiple displacement amplification (MDA). The meaning of the terms “whole genome amplification” and “multiple displacement amplification” are well known in the art and the skilled person is aware of corresponding methods (see for example Lovmar (2006), Hum Mutat. 27(7), 603-14). MDA is known to comprise the use of a highly processive DNA polymerase, for example φ29 DNA polymerase, and random primers for the amplification of genomic DNA. Preferably, WGA and MDA are used for the amplification of the whole genomic DNA of at least one cell, a tissue or an organism. It is also envisioned in context of the method provided herein that genomic DNA of a single cell or a single genome may be amplified, for example by WGA or MDA.

A DNA sample employed herein may preferably have a DNA amount of at least 0.05 ng after said step of amplifying genomic DNA. However, it is also envisaged that lower DNA amounts are to be employed in accordance with the present invention. A person skilled in the art knows the amount of DNA that is to be used, for example, in various genotyping assays.

The DNA sample as described herein and to be selected in context of the method disclosed herein may generally have a wide range of (genomic) DNA concentration. Particularly, DNA samples having a low genomic DNA concentration can be addressed by the provided method with good results.

In a preferred embodiment the DNA sample employed herein obtained after extraction of (genomic) DNA from a biological sample, may have a (genomic) DNA concentration of at least 0.01 pg/μl to 10 μg/μl. Preferably, the genomic DNA concentration may range from at least 0.05 pg/μl to 1 μg/μl. More preferably, the genomic DNA concentration may range from at least 0.1 pg/μl to 100 ng/μl. Even more preferably, the genomic DNA concentration may range from at least 1 pg/μl to 10 ng/μl. Most preferably, the genomic DNA concentration may range from 5 pg/μl to 5 (4.6) ng/μl.

As pointed out above, the DNA sample may be a biological sample or sample comprising organic matter the genomic DNA is derived from. Further, said DNA sample as described herein and in the appended example, particularly blood plasma or blood serum, may particularly be characterized by a low concentration of (genomic) DNA or an overall small amount of (genomic) DNA. For example, said DNA sample may have a (genomic) DNA concentration of less than 100 ng/μl prior to said step of amplifying genomic DNA. Preferably, said DNA sample may have a genomic DNA concentration of less than 90 ng/μl, 80 ng/μl, 70 ng/μl, 60 ng/μl, 50 ng/μl, 40 ng/μl, 30 ng/μl, 20 ng/μl or 10 ng/μl prior to said step of amplifying genomic DNA. More preferably, said DNA sample may have a genomic DNA concentration of less than 9 ng/μl, 8 ng/μl, 7 ng/μl, 6 ng/μl, 5 ng/μl, 4 ng/μl, 3 ng/μl, 2 ng/μl prior to said step of amplifying genomic DNA. Most preferably, said DNA sample may have a genomic DNA concentration of less than 1.15 ng/μl prior to said step of amplifying genomic DNA.

Taken together, the method provided herein is a highly valuable tool for assessing the quality of DNA and therefore suitability of a DNA sample comprising said DNA for genetic analyses, particularly when said DNA sample has been stored during earlier studies which did not particularly bank genomic DNA. The method of the present invention may therefore be particularly useful for assessing the suitability of a sample comprising DNA for genotyping when said DNA (derived from e.g. blood plasma or serum) has been amplified by WGA. The method of the present invention therefore can be seen as a sample selection procedure to ensure reliable DNA quality. The proposed sample selection algorithm is based on a low probability of a high number of homozygotes if several polymorphic markers employed herein, for example STR markers, are genotyped. Since poor DNA quality is associated with genotyping error and genotyping error induces biased association estimates, this extra laboratory effort of genotyping a panel of STR markers might be countervailed by the unbiased invaluable information obtained.

The present invention further relates to a method of genotyping comprising a step of using a DNA sample selected by the corresponding selection method of the present invention and/or a step of applying the selection method in accordance with the present invention.

The present invention further relates to a method for identifying a gene or a locus on a genome, said method comprising a step of using a DNA sample selected by the method of the present invention, a step of applying the selection method of the present invention and/or the step of applying the method of genotyping disclosed herein. In a preferred embodiment of this aspect of the invention, the gene or the locus described above is envisaged to correlate with a certain phenotype. Said phenotype may be a qualitative or quantitative trait and/or a disease or disorder. A non-limiting example for a qualitative trait may be Type 2 diabetes while a quantitative trait may be, for example the (HDL) cholesterol level. The locus described herein may also be a quantitative trait locus (QTL). Non-limiting examples for QTLs in plants may be fruit metabolic QTLs, yield-associated QTLs, biomass QTLs, QTLs for pathogen resistance and the like. Human QTLs may be, for example QTLs for high intelligence, for certain disorders or diseases or for certain features of a human (e.g. BMI (body mass index), blood pressure, total cholesterol, triglycerides, bilirubin, metabolites, wheight, size), and the like.

The present invention also relates to a kit for carrying out the selection method disclosed herein comprising primers for the amplification of the set of polymorphic markers as defined herein. Said kit may be manufactured for use in the selection method as provided herein. The definitions and embodiments given with respect to the mentioned selection method, primers and set of polymorphic markers apply here, mutatis mutandis. In a particularly preferred embodiment of the present invention, the kit (to be prepared in context) of this invention or the methods and uses of the invention may further comprise or be provided with (an) instruction manual(s). For example, said instruction manual(s) may guide the skilled person (how) to select nucleic acid samples, in particular genomic DNA samples in accordance with the present invention. Particularly, said instruction manual(s) may comprise guidance to use or apply the herein provided means, methods and uses.

The kit (to be prepared in context) of this invention may further comprise substances/chemicals and/or equipment suitable/required for carrying out the methods and uses of this invention. Such substances/chemicals and/or equipment may also be solvents, diluents and/or buffers for stabilizing and/or storing (a) compound(s) required for specifically determining the homozygosity of genomic DNA for the set of markers (or subgroup thereof) as defined herein above. Said kit may also comprise substances/chemicals and/or equipment suitable/required for the amplification of such an samples and/or for the reverse transcription of RNA.

The present invention is further described by reference to the following non-limiting figures and examples.

The Figures show:

FIG. 1.

Polymorphic markers with a heterozygosity rate ranging. from 72% to 94%, mean 87(86.8)±7% used for STR genotyping.

The markers D1S495, D2S1338, D3S1314, D5S2498, D8S1130, D11S1983, D12S2078, D19S1167, D20S481 are distributed over 9 different chromosomes. They were chosen from the Genethon and the Cooperative Human Linkage Center Map and are disclosed in the following databases: National Center for Biotechnology (http://www.ncbi.nlm.nih.gov), The GDB Human Genome Database (http://www.gdb.org) and Cooperative Human Linkage Center (http://gai.nci.nih.gov/CHLC).

FIG. 2.

Primers used for STR genotyping markers D1S495, D2S1338, D3S1314, D5S2498, D8S1130, D11S1983, D12S2078, D19S1167, D20S481 on a Biometra T1 PCR system.

The primers were chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

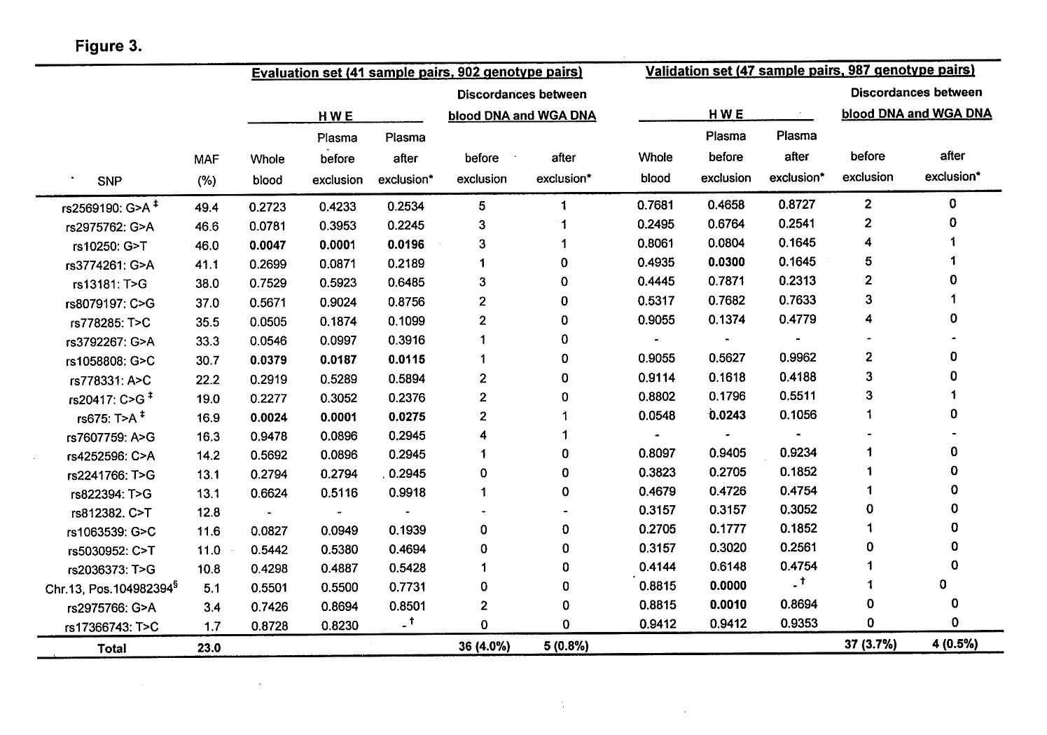

FIG. 3.

Comparison of single nucleotiode polymorphism genotyping in 41 evaluation sample pairs and 47 validation sample pairs of whole blood DNA and whole genome amplified (WGA) plasma DNA.

* Discordances and Hardy Weinberg equilibrium (HWE) were recalculated after exclusion of 12 and 8 samples from the evaluation and the validation set, respectively, with homozygosity at five or more of the nine STR markers genotyped in the WGA plasma samples. short tandem repeats (STR) are listed in FIG. 3.

† After exclusion of samples with homozygosity of five or more of the nine STR marker loci, the remaining samples were homozygous for this SNP. Therefore HWE was not calculable.

‡ genotyped by Taqman assays;

§ exact location: 13 dna: chromosome:NCBI36:13:104982394 (26 Apr. 2007): A>G

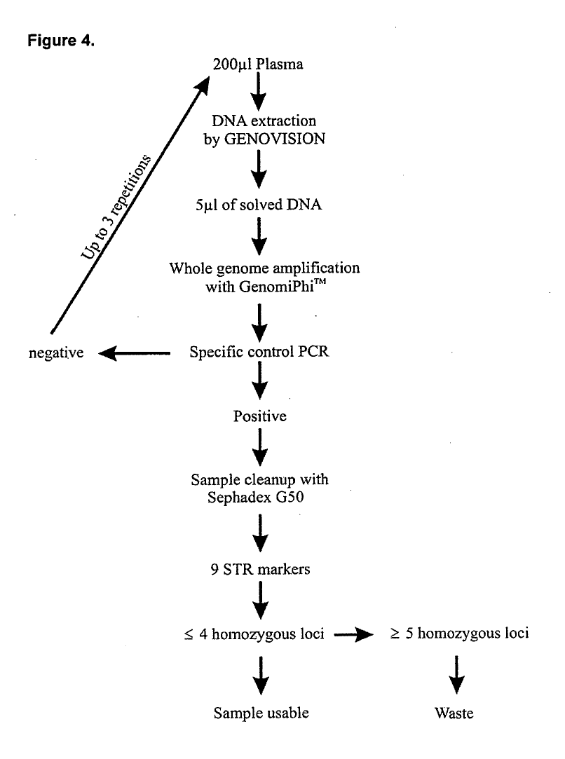

FIG. 4.

Schematic flowchart of sample preparation and evaluation of reliable and non-reliable amplified whole genomic plasma DNA.

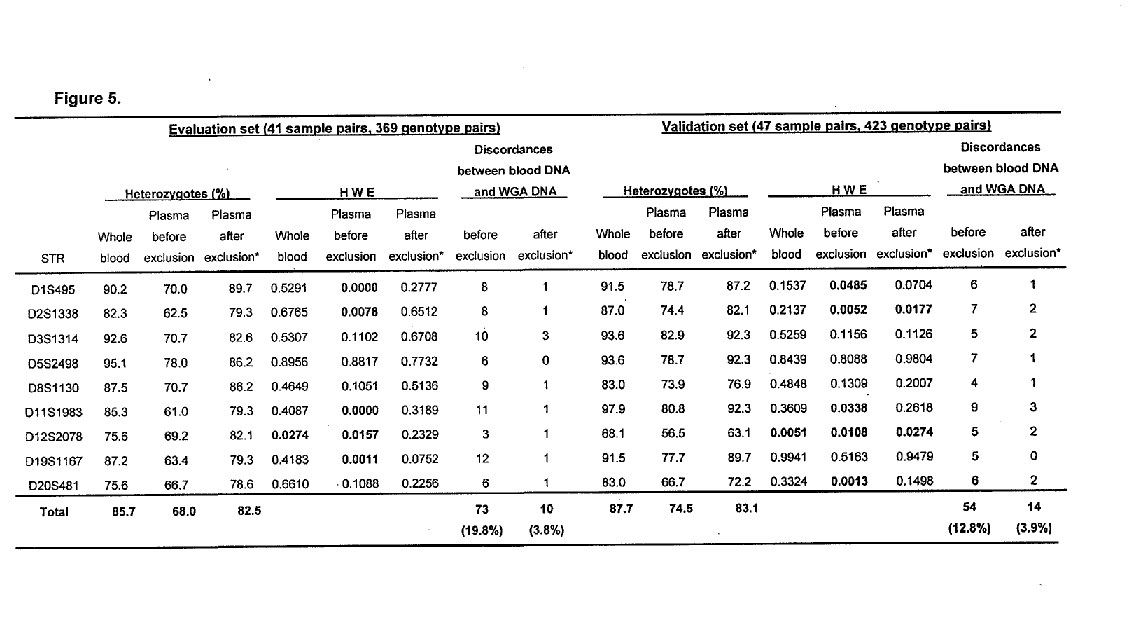

FIG. 5.

Comparison of short tandem repeat (STR) genotyping in 41 evaluation sample pairs and 47 validation sample pairs of whole blood DNA and whole genome amplified (WGA) plasma DNA

* Frequency of heterozygotes, discordances and Hardy Weinberg equilibrium (HWE) were recalculated after exclusion of 12 and 8 samples from the evaluation and the validation set, respectively, with homozygosity at five or more of the nine STR markers genotyped in the WGA plasma samples.

FIG. 6.

Comparison between whole genome amplified plasma DNA (WGA-DNA) and whole blood DNA (gDNA) of the same person.

Panel A and B demonstrate an allelic drop out in the whole genome amplified DNA at loci D1S495 and D5S2498, respectively. DNA samples were analyzed using fluorescent labeled primers with an ABI Prism 3130XL Genetic Analyzer. Panel C and D show an allelic drop out at locus D5S2498 and D2S1338, respectively, analyzed using an 8% PAGE gel.

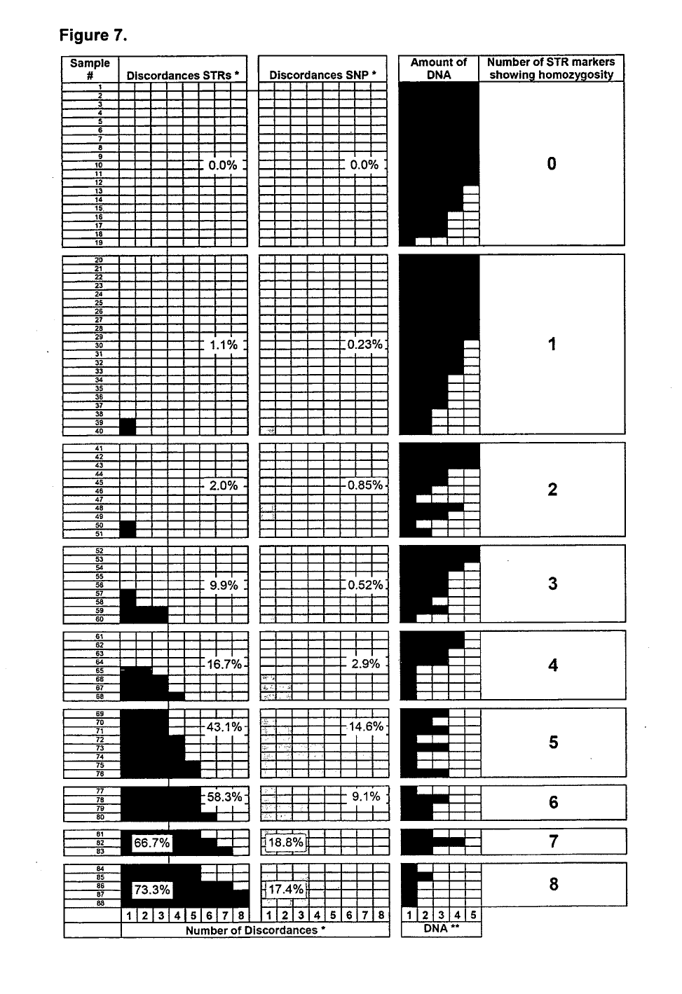

FIG. 7.

Dependency of genotype discordances from the number of short tandem repeats (STR) markers showing homozygosity and the DNA amount used for amplification of whole genomic DNA (WGA).

* colored areas show the number of discordances for STR markers (black) and SNP (single nucleotide polymorphism) markers (grey)

** DNA denotes the amount of DNA used for WGA reaction stratified in classes: 1=≦50.1 ng, 2=0.11-0.20 ng, 3=0.21-0.40 ng, 4=0.41-0.80 ng, 5=>0.80 ng.

The percentages provide the discordances for STRs and SNPs between whole blood DNA and WGA plasma DNA stratified for the number of detected homozygotes for the nine STR markers genotyped in the WGA plasma DNA.

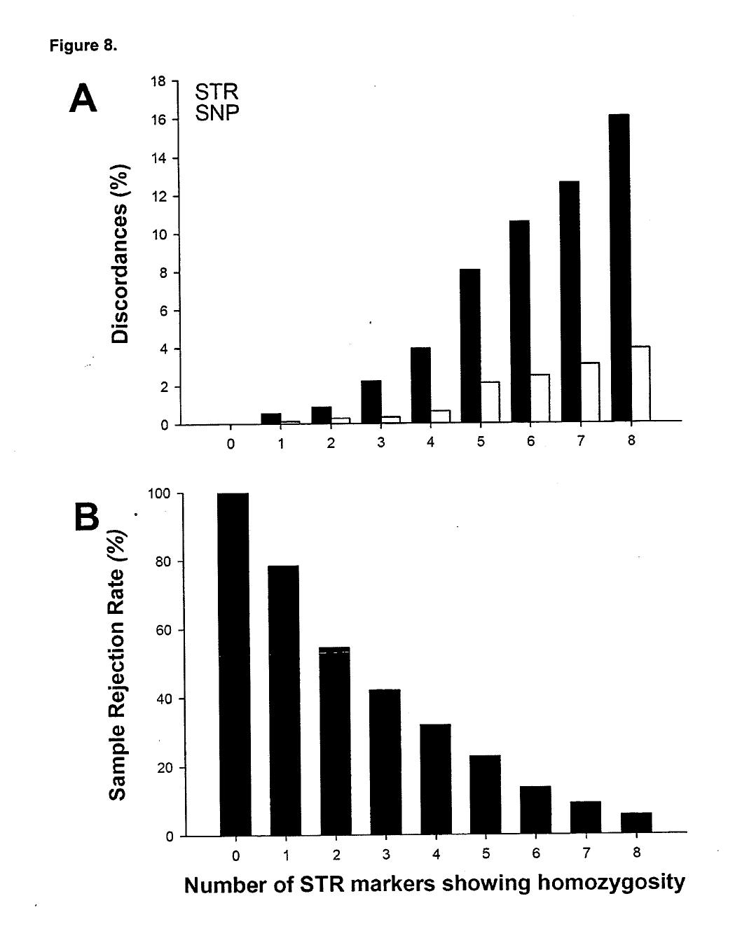

FIG. 8.

Summary of genotyping quality characteristics from genotyping of 88 sample pairs of whole blood DNA and WGA plasma DNA related to the number of detected homozygotes for the nine short tandem repeat (STR) markers genotyped in the whole genomic DNA amplified (WGA) plasma DNA.

The x-axis in each panel represents the number of STR markers showing homozygosity detected in the WGA plasma DNA. Panel A provides the discordance for STRs (black bars) and single nucleotide polymorphism (SNPs) (grey bars) between whole blood DNA and WGA plasma DNA if only samples are considered with up to the respective number of STR markers showing homozygosity. Panel B provides the sample rejection when samples with equal and above the respective number of STR markers showing homozygosity are excluded.

The Example illustrates the invention.

### EXAMPLE 1

### A Sample Selection Algorithm to Improve Quality of Genotyping from Plasma-Derived DNA

### Material and Methods

### Study Population

This study includes patients with end-stage renal disease recruited during the early nineties for an Austrian observational study, see Kronenberg (1995), J Am Soc Nephrol 6, 110-120; Kronenberg (1999), J Am Soc Nephrol 10, 1027-1036. For comparing genotypes from whole blood and plasma DNA, 88 samples were selected with both whole blood and plasma samples available, named “sample pairs”. The 88 sample pairs were divided in two subsets, for the evaluation (development or training) (n=41) and validation (n=47) of the sample selection algorithm, respectively. Genotypes obtained from whole blood DNA were considered as gold standard in this analysis.

Genomic DNA Preparation from Whole Blood and Plasma

DNA from whole blood was isolated from 9 ml of at −80° C. frozen peripheral EDTA blood samples using a standard salting out protocol, see Miller (1988; loc. cit.). Extracted DNA was solved in TE-buffer and stored at −20° C. Genomic plasma DNA was extracted using the fully automated GenoM-48 Robotic Workstation (GENOVISION, Vienna, Austria, Qiagen, Hilden, Germany) according to the manufacturers recommendations. Prior to DNA extraction the 12-year-old and at −80° C. stored plasma samples were defrosted overnight on ice. From each plasma sample 200 μl was put in a 0.5 ml tube and centrifuged 15 minutes with 13000 rpm. The supernatant was discarded and the pellet was used for further processing. The automated extraction method consists of cell lysis using chaotropic reagents, binding of the DNA to silica coated magnetic particles, followed by washing steps and the elution of the pure nucleic acid samples. For all samples the MagAttract DNA Blood M96 Kit (Qiagen, Hilden, Germany) was used. Since only the pellet after the centrifugation of the plasma sample was used, it was assumed that DNA from cells or nuclei harvested from the buffy coat along with the plasma and not the soluble DNA was extracted, which has been documented to circulate in the plasma due to ongoing cell turnover. The extracted DNA was dissolved in 50 μl 10 mMol Tris buffer (pH=8.40) and the samples were stored at −20° C. until use for WGA.

### Whole Genome Amplification (WGA)

The GenomiPhi™ DNA amplification Kit (GE Healthcare, Vienna, Austria) was used for WGA. Briefly, 5 μl of plasma DNA were added to 9 μl GenomiPhi™ sample buffer. This mixture was denaturated at 95° C. for 3 min and then cooled on ice for 1 min. To each sample 10 μl mastermix (containing 9 μl of GenomiPhi™ reaction buffer and 1 μl of GenomiPhi™ enzyme mix) were added and samples were incubated in a PCR thermocycler at 30° C. for 16 hours, followed by a 10 min heating step at 65° C. to inactivate the polymerase. After MDA, the samples were cleaned by spin column chromatography using Millipore MultiScreen-HV Plates filled with Sephadex G-50 according to the manufacturer's protocol. The concentration of the DNA was measured, diluted to 50 ng/μl and then the amplification products were stored at −20° C. After WGA, a specific standard PCR was performed to control the success of the WGA. To exclude contamination, WGA was controlled by a blank sample in each batch as a negative control.

### Determination of DNA Concentration

The concentration and purity of whole blood DNA and WGA plasma DNA was determined with UV-absorbance measurement using the Nanoprop ND-1000 (Nanoprop Technologies, Wilmington, USA). Using high precision pipettes, double measurements in all 88 samples were performed. The average±SD coefficient of variation was 1.17±0.82% with a maximum of 7.3%. Due to the minimal amount of DNA in the plasma samples before WGA, the DNA quantity in these samples was determined by real-time PCR using the Human Quantifiler Kit (Applied Biosystems, Weiterstadt, Germany) according to the manufacturers protocol. Of the solved DNA and the standard 2 μl were pipetted in a 384-well plate, centrifuged and air-dried. Then 5 μl of the Master Mix was added, centrifuged and analyzed on an ABI Prism 7900HT Fast Real-Time PCR System (Applied Biosystems, Weiterstadt, Germany) using 45 cycles.

### STR Genotyping

STR genotyping was performed using an ABI Prism 3130xl Genetic Analyzer using fluorescent-labeled primer pairs. These markers were highly polymorphic with a heterozygosity rate ranging from about 72% to about 94%, mean 87(86.8)±7%. They were chosen from the Genethon and the Cooperative Human Linkage Center Map, and distributed over 9 different chromosomes: D1S495 (SEQ ID No. 1), D2S1338 (SEQ ID No. 2), D3S1314 (SEQ ID No. 3), D5S2498 (SEQ ID No. 4), D8S1130 (SEQ ID No. 5), D11S1983 (SEQ ID No. 6), D12S2078 (SEQ ID No. 7), D19S1167 (SEQ ID No. 8), D20S481 (SEQ ID No. 9), see FIG. 1. Genotyping reactions were performed with 20 ng input DNA in a total volume of 10 μl on a Biometra T1 PCR system using the respective primers as shown in FIG. 2 according to the following PCR protocol: incubation at 95° C. for 15 min, variable cycle number ranging from 27 to 37 at 95° C. for 30 s, 55° C. for 75 s and 72° C. for 30 s and a final extension at 72° C. for 10 min. D3S1314, D11S1983, D19S1167 were done in a single tube reaction, the others were performed in a 2-plex PCR reaction (D1S495 and D5S2498, D2S1338 and D8S1130, D12S2078 and D20S481). The subsequent STR analysis by capillary electrophoresis was performed in three 3-plex reactions mixed together as follows: D1S495, D5S2498 and D11S1983; D2S1338, D8S1130 and D19S1167; D3S1314, D12S2078 and D20S481. 1 μl of PCR product was mixed with 8.5 μl of Hi-Di Formamid and 0.5 μl of ROX 400 HD Size Standard and separated using an ABI PRISM 3130xl Genetic Analyzer (Applied Biosystems). The average lengths of the amplicons was below 200 by in 4 STRs, between 200 and 250 by in 3 STRs and above 250 by in 2 STRs. STR data were analyzed using the Genemapper Software V3.7 (Applied Biosystems). Genotypes were automatically called with predefined parameters and checked manually. For quality control purposes, D1S495, D2S1338, D3S1314, D5S2498 and D12S2078 were also analyzed in the 88 sample pairs on an 8% PAGE Gel, which was stained with ethidiumbromide and visualized on a UV Transilluminator (Herolab, Wiesloch, D).

### MALDI-TOF SNP Genotyping

Genotyping of SNPs was performed using matrix-assisted laser desorption/ionization time-of-flight mass spectrometry (MALDI-TOF MS) to detect allele-specific primer extension products (MassARRAY®, Sequenom, San Diego, Calif.). Genotyping was conducted according to manufacturer's instructions (http://www.sequenom.com/Assets/pdfs/appnotes/hME.pdf, http://www.sequenom.com/Assets/pdfs/appnotes/Multiplexing_hME_App_Note.pdf) with minor modifications, see Weidinger (2004) J Med Genet. 41, 658-663. PCR primers were designed by Sequenom's MassARRAY® AssayDesign 2.0 program. About 95% of the genotypes were automatically called with the SpectroACQUIRE® 3.3.13 software (Sequenom) and 5% were checked manually. The SNPs described in FIG. 3 were genotyped in 4 different multiplex reactions. In total, 20 SNPs were genotyped in the sample pairs of whole blood and plasma DNA samples by MALDI-TOF MS. For technical reasons only 17 of the 20 SNPs were genotyped in both, the evaluation and the validation sample set. Further three SNPs were genotyped only in one of the two sample sets. For quality purposes, the validation sample was genotyped twice.

### TaqMan SNP Genotyping

In addition to the SNPs genotyped by MALDI-TOF, further three SNPs were genotyped using 5′ nuclease allelic discrimination (TaqMan) assays in a 384-well format on the ABI Prism 7900HT Fast Real-Time PCR System (Applied Biosystems, Weiterstadt, Germany) within the Genotyping Unit of the Gene Discovery Core Facility at the Innsbruck Medical University, Austria. PCR primers and probes for the three SNPs were obtained by the Assay-by-design service of Applied Biosystems. 5 ng of air dried DNA were used as template in a reaction volume of 5 μl using TaqMan Universal PCR master mix, primers and probes. The amplification protocol was 95° C. for 10 min followed by 40 cycles of 92° C. for 5 sec and 60° C. for 1 min. Genotypes were called automatically by ABI-PRISM sequence detection system (SDS) software version 2.2.2. Duplicate samples and negative controls were included across the plates to ensure accuracy of genotyping.

### Statistical Analysis

Hardy-Weinberg-Disequilibrium was tested for all SNPs using the program Finetti (http://ihg.gsf.de/cgi-bin/hw/hwa1.pl) and for all STRs using Arlequin (http://lgb.unige.ch/arlequin/).

### Results

### Multiple Displacement Amplification

An overview of the workflow is illustrated in FIG. 4. In a first step, genomic DNA was extracted from 88 plasma samples that were 10-12 years old using magnetic bead technology. The amount of DNA obtained was 250 pg to 230 ng (mean±SD 10.5±28.0 ng) with the concentration ranging from 5 pg/μl to 4.6 ng/μl (mean±SD 213±565 pg/μl). In a second step, the extracted DNA was used for WGA, which yielded an amount of DNA ranging from 1.1 μg to 8.7 μg (mean±SD 4.2±1.3 μg) with a concentration range from 45 ng/μl to 361 ng/μl (mean±SD 156±54 ng/μl). The WGA resulted in an 100- to 67000-fold (mean±SD 13250±15400) amplification of DNA in comparison to the starting material (input DNA).

### Evaluation Sample Set

To test the concordance of the genomic sequence of the whole genome amplified plasma DNA with the genomic sequence of the whole blood DNA, 22 SNPs (19 SNPs using MALDI-TOF MS and 3 SNPs using TaqMan assays) in 41 evaluation sample pairs consisting each of a whole genome-amplified plasma DNA sample and the corresponding whole blood DNA sample from the same person were genotyped. Results of genotyping are listed in FIG. 3. The average call rates were 98.6% and 98.0% for blood DNA and WGA plasma DNA, respectively. In blood and WGA plasma DNA, all SNPs except the same three were in Hardy Weinberg Equilibrium (HWE) (FIG. 3). Comparison of genotypes from whole blood DNA with those from WGA plasma DNA yielded 36 discordances out of 902 genotype pairs. This corresponds to a discordance rate of 4%. Discordances were always allelic drop-outs. No allelic drop-ins were observed. Discordances were observed in only 15 of the 41 sample pairs independent of the SNP genotyping method employed.

A panel of nine highly polymorphic STR markers was used to determine how accurately WGA plasma DNA reproduces highly polymorphic, repetitive sequences compared to the whole blood DNA (FIG. 5). The average call rates were very similar for whole blood DNA and WGA plasma DNA (98.0% and 98.4%, respectively). The observed heterozygosity, however, was markedly lower for WGA plasma DNA compared to whole blood DNA (68.0% vs. 85.7%) pointing to allelic drop-out in the plasma DNA samples. All markers with the exception of D12S2078 (p=0.0274) genotyped from whole blood DNA were within HWE. In contrast, 5/9 STR markers typed from WGA plasma DNA violated HWE. We observed 73 discordant genotypes (discordance 19.8%). All of these were in 18 of the 41 evaluation sample pairs, most of which had already shown discordances in SNP genotyping. The majority of discordances (67 of 73, 91.8%) clustered to 15 samples. The discordances were always allelic drop-outs in the WGA plasma DNA samples compared with a heterozygote genotype of the whole blood DNA. FIGS. 6A and 6B demonstrate the allelic drop-out at two loci, both are from the same sample pair. The same results were obtained when D1S495, D2S1338, D3S1314, D5S2498 and D12S2078 were additionally investigated using 8% PAGE gels (FIGS. 6C and 6D). This was done to exclude method-specific artifacts.

Algorithm to Separate the Wheat from the Chaff

The probability for homozygosity for five different high frequency STRs is far below 1%. Discordances generally showed homozygosity in DNA from plasma samples compared to heterozygosity in whole blood DNA corresponding to allelic dropout in the former. All characteristics of high-quality genotyping improved in the evaluation sample upon exclusion of the 12 samples with five or more homozygosity counts in the WGA plasma DNA: (1) the number of discordant STR genotypes dropped from 73 to 10 corresponding to a pronounced change in the discordance from 19.8% to 3.8% (FIG. 5), (2) an increase of the frequency of heterozygosity of STRs from 68.0% to 82.5% which became similar to the frequency of 85.7% observed in the whole blood DNA (FIG. 5), (3) a drop of the number of HWE violating STRs to zero compared to 5 before exclusion of these samples (FIG. 5), and (4) a pronounced decrease of the discordance among SNPs from 4.0% to 0.8% (FIG. 3).

### Validation Sample Set

The selection algorithm was validated in an independent set of 47 sample pairs using the same set of nine STR markers (FIG. 5) as in the evaluation sample and almost the same set of 21 SNPs (FIG. 3). The algorithm of excluding all samples showing homozygosity at STR markers led to the exclusion of 8 samples. Similar as in the evaluation set, the exclusion of these 8 samples resulted (1) in a drop of the number of discordant STR genotypes from 54 to 14 corresponding to a pronounced change in the discordance from 12.8% to 3.9% (FIG. 5), (2) in an increase in the frequency of heterozygosity of STRs from 74.5% to 83.1% which became similar to the frequency of 87.7% observed in the whole blood DNA (FIG. 5), (3) in a drop of the number of HWE violating STRs to two compared to five before exclusion of these samples (FIG. 5), and (4) in a pronounced decrease of the discordance among SNPs from 3.7% to 0.5% (FIG. 3).

In the same sample, the concordance of a repeated genotyping of the 18 SNPs (1692 genotypes) in whole blood DNA and WGA plasma DNA samples genotyped by MALDI-TOF MS was analyzed. Only 2 samples showed discordances: one discordance (0.12%) was observed between the two whole blood DNA genotypings between the two repeated measures and one discordance (0.12%) between the two repeated WGA plasma DNA genotypings.

Further, it was investigated whether discordances for STR markers are more common for those with longer DNA amplicons. A similar average frequency of discordances of 8.75, 7.7 and 7.5 respectively, was observed in each group of the three amplicon sizes (<200 bp, 200-250 by and >250 bp). This indicates that the lower discordance rate for SNPs may not be explained by the requirement of shorter intact DNA templates used for these assays.

### Determinants of Sample Rejection Rate

FIG. 7 summarizes the results from analyzing the entire set of 88 sample pairs of whole blood DNA and WGA plasma DNA. There are several arguments to exclude all samples with homozygosity at ≧5/9 STR marker in the WGA plasma DNA (or even with homozygosity at ≧4/9 STR marker loci if the criteria should be kept more stringent): it clearly demonstrates that a decreasing amount of DNA extracted from plasma samples and hence less input DNA for the WGA reaction was associated with an increasing number of STR markers showing homozygosity (FIG. 7). Nevertheless several exceptions from this rule can be observed considering samples which showed discordances for STR and SNP genotypings despite an input DNA above 0.2 ng (FIG. 7 sample #48, 49, 69, 71, 73, 76, 78 and 82). Four other samples (#19, 38, 46 and 47) showed no discordances despite <0.2 ng of input DNA. If simply all samples with an input DNA below 0.2 ng had been excluded this would have resulted in a markedly higher sample rejection rate of 32% and still higher frequencies of discordances for STR and SNP markers of 5.6% and 1.48%, respectively, in the remaining samples. FIG. 7 shows that the discordances of STR and SNP genotypes between whole blood DNA and WGA plasma DNA markedly increase to 43.1% and 14.6%, respectively, in case of homozygosity at 5 STR markers detected in the WGA plasma DNA. FIG. 8A demonstrates that the discordance in SNP genotypes can be kept very low (0.63%) when excluding all samples with homozygosity at ≧5/9 STR markers in the WGA plasma DNA. With this threshold this rate is at 3.92% for STR markers. These discordances are markedly lower compared to an exclusion algorithm simply based on the input DNA. And finally, FIG. 8B illustrates the trade-off between accuracy and percentage of rejected samples: upon exclusion of all samples homozygous at ≧5/9 STR loci, 22.7% of the samples were found unsuitable for genotyping. Even more stringent criteria will result in higher exclusion rates.

The present invention refers to the following nucleotide sequences:

### SEQ ID No. 1:

### Nucleotide Sequence of Human STR Marker D1S495.

agctctcaag gacataaaac aagacaagag agaaacttat tctggtattg gtttcagaga
acctcatcaa actttataac tgcccagact tctgggctgt nttgaaaagg tgtgtatagg
gatcctaagc ccatattcta tcctgtgata ccaatctctc tattacagaa caatacagaa
agacaaattt atagancaaa gcacacaaga ttttctacaa cctaagacca gtctcacaaa
tcccttcttc tattaaccaa acctttgcag aggaggcaaa ctctgatgtt taccatgaca
cacacacaca cacacacaca cacacacaca cacacacaca cacacagagg ccagagacct
ggctggtaag gaaatgttta tgctttttga tggcatacca gggtttccag ggttcccttt
ctctgcagct

D1S495 is a human highly polymorphic short tandem repeat (STR) marker which may be used for STR genotyping in accordance with the method of the present invention. The marker was chosen from the Genethon and the Cooperative Human Linkage Center Map and is disclosed in the following databases: National Center for Biotechnology (http://www.ncbi.nlm.nih.gov), The GDB Human Genome Database (http://www.gdb.org) and Cooperative Human Linkage Center (http://gai.nci.nih.gov/CHLC).

### SEQ ID No. 2:

### Nucleotide Sequence of Human STR Marker D2S1338

gtgggaggaa gccagtggat ttggaaacag aaatggcttg gccttgcctg cctgcctgcc
tgcctgcctt ccttccttcc ttccttcctt ccttccttcc ttccttcctt ccctcctgca
atcctttaac ttactgaata actcattatt atgggccncc tgcaggtacc atgctaggta
ctagggatgt aggcatgaac actgacaagg gcctctggga ctggcattct ggtaggaaaa
ggggtgagac agggaagaag ccagcaaatg tatcaacaag aaacagttct aagtgctagg
aagaaatgaa cgtattgatg tcaca

D2S1338 is a human highly polymorphic short tandem repeat (STR) marker which may be used for STR genotyping in accordance with the method of the present invention. The marker was chosen from the Genethon and the Cooperative Human Linkage Center Map and is disclosed in the following databases: National Center for Biotechnology (http://www.ncbi.nlm.nih.gov), The GDB Human Genome Database (http://www.gdb.org) and Cooperative Human Linkage Center (http://gai.nci.nih.gov/CHLC).

### SEQ ID No. 3:

### Nucleotide Sequence of Human STR Marker D3S1314

cctaaaatgg catcttttat gaactttggc anccctttgt aaactttttc tggtcaaaag
caattcatgg aacttgttga taacttacac atttggccct gtgcaaatta agggtctctg
actctgcttc agggaaaacc acacacacac acacacacac acacacacac acacacacac
acacacacac aatgtttttc agggctggaa ggnaaataga gaaaaccaat gactccacag
attgatgatt ccattactca tatggtagac tattctggat tcttggaatc tggagatttt
tacttgcatt ttgatctctc caaaaccttt ctatgactga gttctatgtt tatatgacat
aaaacccaac gagct

D3S1314 is a human highly polymorphic short tandem repeat (STR) marker which may be used for STR genotyping in accordance with the method of the present invention. The marker was chosen from the Genethon and the Cooperative Human Linkage Center Map and is disclosed in the following databases: National Center for Biotechnology (http://www.ncbi.nlm.nih.gov), The GDB Human Genome Database (http://www.gdb.org) and Cooperative Human Linkage Center (http://gai.nci.nih.gov/CHLC).

### SEQ ID No. 4:

### Nucleotide Sequence of Human STR Marker D5S2498

aaagcttaag aaagattgaa tgctatgctg tgtttaagac acttattagt gagacagatt
agatagatag acagtaacat gatagataga tagatagata gatagataga tagatagata
gatagataga tagacagaca gacagacaga tagttagata gatatccctg ttacttgtag
taaattctac catgggactg cctttctttg cactggtgtt tacttataag gtctaaatta
agactagatc ttgtgtcttg cagatctgcg ttttatgaag tcaggaaatt agtaagaaag
ttccccatga tagacttaat atcccaacct ttctctttgg aggattttt

D5S2498 is a human highly polymorphic short tandem repeat (STR) marker which may be used for STR genotyping in accordance with the method of the present invention. The marker was chosen from the Genethon and the Cooperative Human Linkage Center Map and is disclosed in the following databases: National Center for Biotechnology (http://www.ncbi.nlm.nih.gov), The GDB Human Genome Database (http://www.gdb.org) and Cooperative Human Linkage Center (http://gai.nci.nih.gov/CHLC).

### SEQ ID No. 5:

### Nucleotide Sequence of Human STR Marker D8S1130

cccacaggca ttcaggaggt tgtacatcac aaaagagata aatcaagact aacagcataa
tgaactgttg tttgggggaa tttaaccatc tgattctaaa atctgtatgg aaatgaaagg
nnccnnannt agccatgnca ntcacacaca cagttangat aagtgggaag atttggctct
gttggagaca gnctcataga tagatagata gatagataga tagatagata gatagataga
tagatagatg tntagataga tctgattgag aagtttatta acttcattat gaaagctata
gcagtaagac agcatngggc cntttnggtn ccaaggttnt tggncccant tnggnnccnn
tgnctttttn ggnc

D8S1130 is a human highly polymorphic short tandem repeat (STR) marker which may be used for STR genotyping in accordance with the method of the present invention. The marker was chosen from the Genethon and the Cooperative Human Linkage Center Map and is disclosed in the following databases: National Center for Biotechnology (http://www.ncbi.nlm.nih.gov), The GDB Human Genome Database (http://www.gdb.org) and Cooperative Human Linkage Center (http://gai.nci.nih.gov/CHLC).

### SEQ ID No. 6:

### Nucleotide Sequence of Human STR Marker D11S1983

gggtgacaga atgagattct gtgtctaaaa acagaaaaga atagagatag atagatgata
gatagataga tagatagata gatagataga tagatagata gatagataga taataggtaa
aagatagata gataatagat gatagatgat agatagatag atgatagata gatagataga
tagatagata gatagataga ttcattggtt gactttnatt cccctctttc ctggtaaaat
atttggtgat tcctgatttc ctttttatct ttgagtttgg aaattccnat tcatacttaa
ganaatgnct gttgcatgcc ccnngtattc t

D11S1983 is a human highly polymorphic short tandem repeat (STR) marker which may be used for STR genotyping in accordance with the method of the present invention. The marker was chosen from the Genethon and the Cooperative Human Linkage Center Map and is disclosed in the following databases: National Center for Biotechnology (http://www.ncbi.nlm.nih.gov), The GDB Human Genome Database (http://www.gdb.org) and Cooperative Human Linkage Center (http://gai.nci.nih.gov/CHLC).

### SEQ ID No. 7:

### Nucleotide Sequence of Human STR Marker D12S2078

agcccataat taaaataaat tattgagcac caaaaagtta cagattgatt agcacaattt
cacgtacttg gcaagcaggg aaaaatccgg gctgaaatga aagggcaggg ggcacaagac
tgctgagaac tggaaccatc aattgaacct gttttagtaa attattctga ccttttgaaa
tcttccaatt ggtgatcaaa tatctctata tctatctatc tatttatcta tctatctatc
tatctatcta tctatgtatc cacacacata aatgtcataa aaagaaggat ggggatgagg
gattctgacc tttagagtta acagnaat

D12S2078 is a human highly polymorphic short tandem repeat (STR) marker which may be used for STR genotyping in accordance with the method of the present invention. The marker was chosen from the Genethon and the Cooperative Human Linkage Center Map and is disclosed in the following databases: National Center for Biotechnology (http://www.ncbi.nlm.nih.gov), The GDB Human Genome Database (http://www.gdb.org) and Cooperative Human Linkage Center (http://gai.nci.nih.gov/CHLC).

### SEQ ID No. 8:

### Nucleotide Sequence of Human STR Marker D19S1167

ccatattgtc cctcaaagtg gctctactaa tatacattac caccaacagt gtacaaaggt
tccatggggg attctttcag attgagtggt cagagagggc ttctttgaga aggcttctga
gagttggcca tgagaaggtg tggtgagaag ccttctcagc tgagggaaca gcaaggtaaa
aggnccggag tgagaaagca aacttggcac cttccgggag caagcaggca ttgcntctna
taatgtttcc tcctggtaaa atgacagctc tgggagggca gggttcttat ttcctatcta
tctatctatc atctatctat ctatctatct atctatctat ctatctatct atctatctat
catctatcat ctatctatct atctatctat catctatcta tctatcatct atctatctat
ctatatctat ctgtctatct aatcacctat ctttctatgt atctatcatc tatgtatcta
tccatctgtc tttatttatt tatagagtca gagtcttgct ctgttgccca ttgta

D19S1167 is a human highly polymorphic short tandem repeat (STR) marker which may be used for STR genotyping in accordance with the method of the present invention. The marker was chosen from the Genethon and the Cooperative Human Linkage Center Map and is disclosed in the following databases: National Center for Biotechnology (http://www.ncbi.nlm.nih.gov), The GDB Human Genome Database (http://www.gdb.org) and Cooperative Human Linkage Center (http://gai.nci.nih.gov/CHLC).

### SEQ ID No. 9:

### Nucleotide Sequence of Human STR Marker D20S481

cagtaatggt gagaaatggg ttatgagtgc acacaggaaa attggtacct gatgtggaag
gtagagaaat actgaggaaa aagctctctg aagcaggtgt tctatctatc tatctatcta
tctatctatc tatctatcta tctatctatc tatctatcat ctgtctatca tcaacatcat
catcatcatc ctttcctctc tttctagtgc aatctgtggt tacctcttag ctgtgtgtct
ttttgctgtt ttatgacaca aagccggaaa gttgatatat ctntgaggta ggacagtaaa
ggtatatntn nt

D20S481 is a human highly polymorphic short tandem repeat (STR) marker which may be used for STR genotyping in accordance with the method of the present invention. The marker was chosen from the Genethon and the Cooperative Human Linkage Center Map and is disclosed in the following databases: National Center for Biotechnology (http://www.ncbi.nlm.nih.gov), The GDB Human Genome Database (http://www.gdb.org) and Cooperative Human Linkage Center (http://gai.nci.nih.gov/CHLC).

### SEQ ID No. 10:

Nucleotide sequence of first primer used for genotyping STR marker D1S495. The primer may be used for detecting a Di repeat type of said STR marker.

5′-(Fam)-ACCAAACCTTTGCAGAGGA-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 11:

Nucleotide sequence of second primer used for genotyping STR marker D1S495. The primer may be used for detecting a Di repeat type of said STR marker.

5′-AACCCTGGTATGCCATCA-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 12:

Nucleotide sequence of first primer used for genotyping STR marker D2S1338. The primer may be used for detecting a Tetra repeat type of said STR marker.

5′-(Fam)-CCAGTGGATTTGGAAACAGA-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 13:

Nucleotide sequence of second primer used for genotyping STR marker D2S1338. The primer may be used for detecting a Tetra repeat type of said STR marker.

5′-ACCTAGCATGGTACCTGCAG-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 14:

Nucleotide sequence of first primer used for genotyping STR marker D3S1314. The primer may be used for detecting a Di repeat type of said STR marker.

5′-(Hex)-AACTTACACATTTGGCCCTG-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 15:

Nucleotide sequence of second primer used for genotyping STR marker D3S1314. The primer may be used for detecting a Di repeat type of said STR marker.

5′-TCAATCTGTGGAGTCATTGG-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 16:

Nucleotide sequence of first primer used for genotyping STR marker D5S2498. The primer may be used for detecting a Tetra repeat type of said STR marker.

5′-(Fam)-AATGCTATGCTGTGTTTAAGACA-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 17:

Nucleotide sequence of second primer used for genotyping STR marker D5S2498. The primer may be used for detecting a Tetra repeat type of said STR marker.

5′-AAAACGCAGATCTGCAAGAC-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 18:

Nucleotide sequence of first primer used for genotyping STR marker D8S1130. The primer may be used for detecting a Tetra repeat type of said STR marker.

5′-Fam-GAAGATTTGGCTCTGTTGGA-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 19:

Nucleotide sequence of second primer used for genotyping STR marker D8S1130. The primer may be used for detecting a Tetra repeat type of said STR marker.

5′-TGTCTTACTGCTATAGCTTTCATAA-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 20:

Nucleotide sequence of first primer used for genotyping STR marker D11S1983. The primer may be used for detecting a Tetra repeat type of said STR marker.

5′-Hex-ATTCTGTGTCTAAAAACAGAAAAGA-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 21:

Nucleotide sequence of second primer used for genotyping STR marker D11S1983. The primer may be used for detecting a Tetra repeat type of said STR marker.

5′-TTACCAGGAAAGAGGGGAAT-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 22:

Nucleotide sequence of first primer used for genotyping STR marker D12S2078. The primer may be used for detecting a Tetra repeat type of said STR marker.

5′-(Fam)-ATTTCACGTACTTGGCAAGC-3′

The primers were chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 23:

Nucleotide sequence of second primer used for genotyping STR marker D12S2078. The primer may be used for detecting a Tetra repeat type of said STR marker.

5′-AAGGTCAGAATCCCTCATCC-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 24:

Nucleotide sequence of first primer used for genotyping STR marker D1951167. The primer may be used for detecting a Tetra repeat type of said STR marker.

5′-Fam-CTGAGGGAACAGCAAGGTAA-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 25:

Nucleotide sequence of second primer used for genotyping STR marker D19S1167. The primer may be used for detecting a Tetra repeat type of said STR marker.

5′-AGAGCAAGACTCTGACTCTATAAAT-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 26:

Nucleotide sequence of first primer used for genotyping STR marker D20S481. The primer may be used for detecting a Tetra repeat type of said STR marker.

5′-Fam-TGGGTTATGAGTGCACACAG-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

### SEQ ID No. 27:

Nucleotide sequence of second primer used for genotyping STR marker D20S4815. The primer may be used for detecting a Tetra repeat type of said STR marker.

5′-AACAGCAAAAAGACACACAGC-3′

The primer was chosen from the Genethon and the Cooperative Human Linkage Center Map and are disclosed in the following databases: National Center for Biotechnology (http://www.ncbi.nlm.nih.gov), The GDB Human Genome Database (http://www.gdb.org), Cooperative Human Linkage Center (http://gai.nci.nih.gov/CHLC).

1. A method for selecting a DNA sample comprising genomic DNA suitable for genotyping, said method comprising the steps of:
(i) pre-genotyping said genomic DNA using a set of polymorphic markers;
(ii) determining out of said set of polymorphic markers the percentage of polymorphic markers for which said genomic DNA is homozygous; and
(iii) selecting said DNA sample when said genomic DNA is homozygous for less than 70% of said set of polymorphic markers.

2. The method of claim 1, wherein in step (iii) said genomic DNA is homozygous for less than 60% of said set of polymorphic markers DNA.

3. The method of claim 1, wherein said DNA sample is derived from a mammal.

4. The method of claim 3, wherein said mammal is a human.

5. The method of claim 1, wherein said DNA sample is derived from or is (a) cell(s), (a) tissue(s) or (a) body fluid(s).

6. The method of claim 5, wherein said body fluid is blood plasma or blood serum.

7. The method of claim 1, further comprising the step of amplifying said genomic DNA from said DNA sample prior to said pre-genotyping.

8. The method of claim 7, wherein said step of amplifying genomic DNA comprises multiple displacement amplification (MDA).

9. The method of claim 1, wherein said DNA sample has a genomic DNA concentration of less than 1.15 ng/μl prior to said step of amplifying genomic DNA.

10. The method of claim 1, wherein said genomic DNA comprises the whole genomic DNA.

11. The method of claim 1, wherein said set of polymorphic markers is a set of short tandem repeat (STR) markers or single nucleotide polymorphism (SNP) markers.

12. The method of claim 1, wherein said set of polymorphic markers comprises at least 2, 3, 6 or 9 polymorphic markers.

13. The method of claim 1, wherein the probability for homozygosity of the genomic DNA for all markers of said set of polymorphic markers is less than 2%.

14. The method of claim 1, wherein said set of polymorphic markers comprises at least 5 polymorphic markers, and wherein the average heterozygozity rate of said 5 polymorphic markers is at least 87%.

15. The method of claim 13, wherein said set of polymorphic markers is one or more polymorphic marker selected from the group consisting of D1S495, D2S1338, D3S1314, D5S2498, D8S1130, D11S1983, D12S2078, D19S1167, and D20S481.

16. The method of claim 1, wherein said DNA sample is selected, when said genomic DNA is homozygous for less than 56% of said set of polymorphic markers.

17. The method of claim 1, wherein said DNA sample is selected, when said genomic DNA is homozygous for less than 50% of said set of polymorphic markers.

18. The method of claim 1, wherein said DNA sample is selected, when said genomic DNA is homozygous for less than 45% of said set of polymorphic markers.

19. The method of claim 1, wherein said DNA sample is selected, when said genomic DNA is homozygous for less than 40% of said set of polymorphic markers.

20. The method of claim 1, wherein said genotyping is single nucleotide polymorphism (SNP) genotyping or short tandem repeat (STR) genotyping

21. The method of claim 1, wherein said pre-genotyping is SNP genotyping or STR genotyping.

22. The method of claim 1, further comprising a step of using said DNA sample for genotyping.

23. The method of claim 1, further comprising a step of using said DNA sample to identify a gene or a locus on a genome.

24. The method of claim 23, wherein said gene or said locus correlates with a certain phenotype.

25. The method of claim 24, wherein said phenotype is a qualitative or quantitative trait.

26. The method of claim 24, wherein said phenotype is a disease or disorder.

27. The method of claim 23, wherein said locus is a quantitative trait locus.

28. A kit comprising primers for the amplification of the set of polymorphic markers as defined claim 1.

29. The method of claim 14, wherein said set of polymorphic markers is one or more polymorphic marker selected from the group consisting of D1S495, D2S1338, D3S1314, D5S2498, D8S1130, D11S1983, D12S2078, D19S1167, and D20S481.

30. The method of claim 25, wherein said phenotype is a disease or disorder.

31. The method of claim 28, wherein said set of polymorphic markers is one or more polymorphic marker selected from the group consisting of D1S495, D2S1338, D3S1314, D5S2498, D8S1130, D11S1983, D12S2078, D19S1167, and D20S481.


SEQUENCE LISTING (27 sequences)

SEQ ID NO: 1 - DNA, 430 bp, homo sapiens
  misc_feature: STR marker D1S495
agctctcaaggacataaaacaagacaagagagaaacttattctggtattggtttcagagaacctcatcaaactttataactgcccagacttctgggctgtnttgaaaaggtgtgtatagggatcctaagcccatattctatcctgtgataccaatctctctattacagaacaatacagaaagacaaatttatagancaaagcacacaagattttctacaacctaagaccagtctcacaaatcccttcttctattaaccaaacctttgcagaggaggcaaactctgatgtttaccatgacacacacacacacacacacacacacacacacacacacacacacacacagaggccagagacctggctggtaaggaaatgtttatgctttttgatggcataccagggtttccagggttccctttctctgcagct

SEQ ID NO: 2 - DNA, 325 bp, homo sapiens
  misc_feature: STR marker D2S1338
gtgggaggaagccagtggatttggaaacagaaatggcttggccttgcctgcctgcctgcctgcctgccttccttccttccttccttccttccttccttccttccttccttccctcctgcaatcctttaacttactgaataactcattattatgggccncctgcaggtaccatgctaggtactagggatgtaggcatgaacactgacaagggcctctgggactggcattctggtaggaaaaggggtgagacagggaagaagccagcaaatgtatcaacaagaaacagttctaagtgctaggaagaaatgaacgtattgatgtcaca

SEQ ID NO: 3 - DNA, 375 bp, homo sapiens
  misc_feature: STR marker D3S1314
cctaaaatggcatcttttatgaactttggcanccctttgtaaactttttctggtcaaaagcaattcatggaacttgttgataacttacacatttggccctgtgcaaattaagggtctctgactctgcttcagggaaaaccacacacacacacacacacacacacacacacacacacacacacacacacacaatgtttttcagggctggaaggnaaatagagaaaaccaatgactccacagattgatgattccattactcatatggtagactattctggattcttggaatctggagatttttacttgcattttgatctctccaaaacctttctatgactgagttctatgtttatatgacataaaacccaacgagct

SEQ ID NO: 4 - DNA, 349 bp, homo sapiens
  source: STR marker D5S2498
aaagcttaagaaagattgaatgctatgctgtgtttaagacacttattagtgagacagattagatagatagacagtaacatgatagatagatagatagatagatagatagatagatagatagatagatagatagacagacagacagacagatagttagatagatatccctgttacttgtagtaaattctaccatgggactgcctttctttgcactggtgtttacttataaggtctaaattaagactagatcttgtgtcttgcagatctgcgttttatgaagtcaggaaattagtaagaaagttccccatgatagacttaatatcccaacctttctctttggaggattttt

SEQ ID NO: 5 - DNA, 374 bp, homo sapiens
  misc_feature: STR marker D8S1130
cccacaggcattcaggaggttgtacatcacaaaagagataaatcaagactaacagcataatgaactgttgtttgggggaatttaaccatctgattctaaaatctgtatggaaatgaaaggnnccnnanntagccatgncantcacacacacagttangataagtgggaagatttggctctgttggagacagnctcatagatagatagatagatagatagatagatagatagatagatagatagatagatgtntagatagatctgattgagaagtttattaacttcattatgaaagctatagcagtaagacagcatngggccntttnggtnccaaggttnttggncccanttnggnnccnntgnctttttnggnc

SEQ ID NO: 6 - DNA, 331 bp, homo sapiens
  misc_feature: STR marker D11S1983
gggtgacagaatgagattctgtgtctaaaaacagaaaagaatagagatagatagatgatagatagatagatagatagatagatagatagatagatagatagatagatagataataggtaaaagatagatagataatagatgatagatgatagatagatagatgatagatagatagatagatagatagatagatagatagattcattggttgactttnattcccctctttcctggtaaaatatttggtgattcctgatttcctttttatctttgagtttggaaattccnattcatacttaaganaatgnctgttgcatgccccnngtattct

SEQ ID NO: 7 - DNA, 328 bp, homo sapiens
  misc_feature: STR marker D12S2078
agcccataattaaaataaattattgagcaccaaaaagttacagattgattagcacaatttcacgtacttggcaagcagggaaaaatccgggctgaaatgaaagggcagggggcacaagactgctgagaactggaaccatcaattgaacctgttttagtaaattattctgaccttttgaaatcttccaattggtgatcaaatatctctatatctatctatctatttatctatctatctatctatctatctatctatgtatccacacacataaatgtcataaaaagaaggatggggatgagggattctgacctttagagttaacagnaat

SEQ ID NO: 8 - DNA, 535 bp, homo sapiens
  misc_feature: STR marker D19S1167
ccatattgtccctcaaagtggctctactaatatacattaccaccaacagtgtacaaaggttccatgggggattctttcagattgagtggtcagagagggcttctttgagaaggcttctgagagttggccatgagaaggtgtggtgagaagccttctcagctgagggaacagcaaggtaaaaggnccggagtgagaaagcaaacttggcaccttccgggagcaagcaggcattgcntctnataatgtttcctcctggtaaaatgacagctctgggagggcagggttcttatttcctatctatctatctatcatctatctatctatctatctatctatctatctatctatctatctatctatcatctatcatctatctatctatctatctatcatctatctatctatcatctatctatctatctatatctatctgtctatctaatcacctatctttctatgtatctatcatctatgtatctatccatctgtctttatttatttatagagtcagagtcttgctctgttgcccattgta

SEQ ID NO: 9 - DNA, 312 bp, homo sapiens
  misc_feature: STR marker D20S481
cagtaatggtgagaaatgggttatgagtgcacacaggaaaattggtacctgatgtggaaggtagagaaatactgaggaaaaagctctctgaagcaggtgttctatctatctatctatctatctatctatctatctatctatctatctatctatctatcatctgtctatcatcaacatcatcatcatcatcctttcctctctttctagtgcaatctgtggttacctcttagctgtgtgtctttttgctgttttatgacacaaagccggaaagttgatatatctntgaggtaggacagtaaaggtatatntnnt

SEQ ID NO: 10 - DNA, 19 bp, artificial sequence
  source: /note = “Description of artificial sequence primer”
accaaacctttgcagagga

SEQ ID NO: 11 - DNA, 18 bp, artificial sequence
  source: /note = “Description of artificial sequence primer”
aaccctggtatgccatca

SEQ ID NO: 12 - DNA, 20 bp, artificial sequence
  source: /note = “Description of artificial sequence primer”
ccagtggatttggaaacaga

SEQ ID NO: 13 - DNA, 20 bp, artificial sequence
  source: /note = “Description of artificial sequence primer”
acctagcatggtacctgcag

SEQ ID NO: 14 - DNA, 20 bp, artificial sequence
  source: /note = “Description of artificial sequence primer”
aacttacacatttggccctg

SEQ ID NO: 15 - DNA, 20 bp, artificial sequence
  source: /note = “Description of artificial sequence primer”
tcaatctgtggagtcattgg

SEQ ID NO: 16 - DNA, 23 bp, artificial sequence
  source: /note = “Description of artificial sequence primer”
aatgctatgctgtgtttaagaca

SEQ ID NO: 17 - DNA, 20 bp, artificial sequence
  source: /note = “Description of artificial sequence primer”
aaaacgcagatctgcaagac

SEQ ID NO: 18 - DNA, 20 bp, artificial sequence
  source: /note = “Description of artificial sequence primer”
gaagatttggctctgttgga

SEQ ID NO: 19 - DNA, 25 bp, artificial sequence
  source: /note = “Description of artificial sequence primer”
tgtcttactgctatagctttcataa

SEQ ID NO: 20 - DNA, 25 bp, artificial sequence
  source: /note = “Description of artificial sequence primer”
attctgtgtctaaaaacagaaaaga

SEQ ID NO: 21 - DNA, 20 bp, artificial sequence
  source: /note = “Description of artificial sequence primer”
ttaccaggaaagaggggaat

SEQ ID NO: 22 - DNA, 20 bp, artificial sequence
  source: /note = “Description of artificial sequence primer”
atttcacgtacttggcaagc

SEQ ID NO: 23 - DNA, 20 bp, artificial sequence
  source: /note = “Description of artificial sequence primer”
aaggtcagaatccctcatcc

SEQ ID NO: 24 - DNA, 20 bp, artificial sequence
  source: /note = “Description of artificial sequence primer”
ctgagggaacagcaaggtaa

SEQ ID NO: 25 - DNA, 25 bp, artificial sequence
  source: /note = “Description of artificial sequence primer”
agagcaagactctgactctataaat

SEQ ID NO: 26 - DNA, 20 bp, artificial sequence
  source: /note = “Description of artificial sequence primer”
tgggttatgagtgcacacag

SEQ ID NO: 27 - DNA, 21 bp, artificial sequence
  source: /note = “Description of artificial sequence primer”
aacagcaaaaagacacacagc